(19) United States
(12) Patent Application Publication
(10) Pub. No.: US 2024/0324957 A1
(43) Pub. Date: Oct. 3, 2024

(54) FINGER WEARABLE DEVICES AND METHODS FOR PRODUCING FINGER WEARABLE DEVICES

(71) Applicant: MOVANO INC., Pleasanton, CA (US)

(72) Inventors: Hector Realubit, North Hollywood, CA (US); Gabriel Cohn, Mill Valley, CA (US); Michael A. Leabman, Pleasanton, CA (US)

(21) Appl. No.: 18/622,763

(22) Filed: Mar. 29, 2024

Related U.S. Application Data
(60) Provisional application No. 63/456,140, filed on Mar. 31, 2023.
(60) Provisional application No. 63/530,374, filed on Aug. 2, 2023.

(51) Int. Cl. A61B 5/00; A61B 5/024; A61B 5/1455

(52) U.S. Cl. CPC A61B 5/6826; A61B 5/02427; A61B 5/1455; A61B 2560/0204; A61B 2562/164

(57) ABSTRACT

Finger wearable devices for health monitoring and methods for producing such devices are disclosed. A device includes an outer circular piece having two opposing ends, an inner circular piece having two opposing ends, sensor electronics including a PCB and electronic components, the sensor electronics encapsulated in a cavity between the outer and inner circular pieces, the outer circular piece and the inner circular piece include attachment features that are complementary to each other and configured to enable the outer circular piece and the inner circular piece to mate together at the attachment features such that the outer circular piece and the inner circular piece are held together by the attachment features and such that the outer circular piece and the inner circular piece create the cavity within which the sensor electronics are encapsulated, and an encapsulant in the cavity between the outer circular piece and the inner circular piece.

### CROSS-REFERENCE TO RELATED APPLICATION

This application claims priority to provisional U.S. Patent Application Ser. No. 63/456,140, filed Mar. 31, 2023, and provisional U.S. Patent Application Ser. No. 63/530,374, filed Aug. 2, 2023, which are incorporated by reference herein.

### BACKGROUND

Wearable devices that monitor activity and/or health parameters exist in various forms including wrist worn devices and finger worn devices. Whether the device is worn on the wrist or a finger, it is important the device fit correctly for both comfort and performance. Wrist worn devices typically have adjustable wristbands but finger worn devices, such as rings also known as smart rings, have typically been produced in different sizes of rings to fit different sizes of fingers. While producing a range of sizes for smart rings provides options for different sized fingers, the typical smart rings can be difficult to put on and take off and are not able to adapt to changes in finger size due to, for example, temporary swelling.

### BRIEF DESCRIPTION OF THE DRAWINGS

FIG. 1 is an expanded view of an example of a wearable device that includes an outer circular metal piece, an inner circular plastic piece, and sensor electronics.

FIG. 2A depicts an expanded view of an example of the attachment features at the two opposing ends of the inner circular plastic piece.

FIG. 2B depicts an example of the attachment features at the two opposing ends of the outer circular metal piece.

FIG. 2C is a side cutaway view of an assembled ring that shows complementary attachment features located at the sides of the outer circular metal piece and at the sides of the inner circular plastic piece.

FIG. 3 illustrates an example assembly process of the wearable device of FIG. 1.

FIG. 4 is a side cutaway view of an example of a fully assembled wearable ring, which includes the outer circular metal piece, the inner circular plastic piece, and the sensor electronics as described with reference to FIGS. 1-3.

FIG. 5A is a side view of the sensor electronics that depicts a PCB that includes a first section and a second section in which the second section of the PCB is thinner than the first section of the PCB.

FIG. 5B is a plan view of the outer surface of the PCB that shows the various IC devices and the battery relative to the first and second sections of the PCB.

FIG. 5C is a plan view of the outer surface of the PCB without any electronic components attached thereto, which shows an example of various interfaces of the PCB that enable attachment of the electronic components to the PCB.

FIG. 5D is a plan view of the inner surface of the PCB that shows the battery charging contacts, two LED banks, and the photodetector relative to the first and second sections of the PCB.

FIG. 5E is a plan view of the inner surface of the PCB without any electronic components attached thereto, which shows the interfaces that enable attachment of the electronic components to the PCB.

FIG. 6A depicts an example angular relationship between two LED banks and a photodetector in which the LED banks are at approximately 50 degrees relative to a horizontal axis on which the photodetector sits.

FIG. 6B is a side cutaway view of an example of a fully assembled wearable ring, which includes an outer circular metal piece, an inner circular plastic piece, and sensor electronics that includes a photodetector and two LED banks.

FIG. 7A is a plan view of the inner surface of a PCB that shows the battery charging contacts, two LED banks, the photodetector, and two unused LED bank interfaces relative to the first and second sections of the PCB.

FIG. 7B is a plan view of the inner surface of the PCB without any electronic components attached thereto, which shows the interfaces that enable attachment of the electronic components to the PCB.

FIG. 8A is an example of an LED bank that may be used in a wearable health monitoring device, including a wearable health monitoring device that is intended to be worn on a finger as a ring.

FIG. 8B depicts an example of a combined LED and photodetector packaged into a single device.

FIG. 9 is an example computing system that includes a sensor system, a processor, memory, a communications interface, a battery, a user interface device, a tactile indicator device, and a speaker.

FIGS. 10A-10E are perspective views of the inner circular plastic piece.

FIGS. 11A and 11B are perspective views of the inner circular plastic piece.

FIGS. 12A and 12B are perspective views of the outer circular metal piece.

FIG. 12C is a cross section view of the outer circular metal piece.

Throughout the description, similar reference numbers may be used to identify similar elements.

### SUMMARY

Finger wearable devices and methods for producing finger wearable devices are disclosed. In an example, a finger wearable device for health monitoring includes an outer circular piece, the outer circular piece having two opposing ends, an inner circular piece, the inner circular piece having two opposing ends, sensor electronics including a printed circuit board (PCB) and electronic components connected to the PCB, the sensor electronics encapsulated in a cavity between the outer circular piece and the inner circular piece, wherein the outer circular piece and the inner circular piece include attachment features that are complementary to each other and configured to enable the outer circular piece and the inner circular piece to mate together at the attachment features such that the outer circular piece and the inner circular piece are held together by the attachment features and such that the outer circular piece and the inner circular piece create the cavity within which the sensor electronics are encapsulated, and an encapsulant in the cavity between the outer circular piece and the inner circular piece.

Other aspects and advantages of embodiments of the present invention will become apparent from the following detailed description taken in conjunction with the accompanying drawings.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

A finger wearable device, or ring, or smart ring, includes an outer circular metal piece, an inner circular plastic piece, and sensor electronics enclosed within a cavity formed by the outer circular metal piece and the inner circular plastic piece. The finger wearable device does not form a complete monolithic circle or ring. Rather, the outer circular metal piece and the inner circular plastic piece include opposing ends that enable the finger wearable device to be designed with some flexibility, e.g., such that the opposing ends of the wearable device are able to move or “flex” relative to each other in response to typical forces encountered from putting the wearable device on a finger, taking the wearable device off of a finger, and/or in response to changes in the size of a finger (e.g., due to swelling). The flexibility of the finger wearable device can enable a user to wear a tighter fitting device in a typical position below a knuckle than would be possible with a finger ring that has no ability to flex. It has been found that a tighter fitting finger wearable device provides more consistent skin to device contact, which can result in better signal generation for an embedded sensor such as an optical and/or radio frequency (RF) sensor.

In an embodiment, a finger wearable device that includes an outer circular metal piece and an inner circular plastic piece can be snapped together to enclose sensor electronics within a cavity between the two pieces. The cavity can then be filled with an encapsulant material to further secure the attachment between the two pieces and to protect the sensor electronics. Because both the inner circular plastic piece and the outer circular metal piece are open-ended, one or both of the pieces can be flexed to help fit the two pieces together in an assembly process. In one example, the two pieces have complimentary attachment features at the ends of the pieces that enable the two pieces to fit snuggly together with the application of enough force to flex at least one of the two pieces. Thus, in addition to the benefits around user comfort and sensor performance provided by the open-ended ring design, the open-ended ring design also enables design enhancements and corresponding assembly processes that heretofore have not existed.

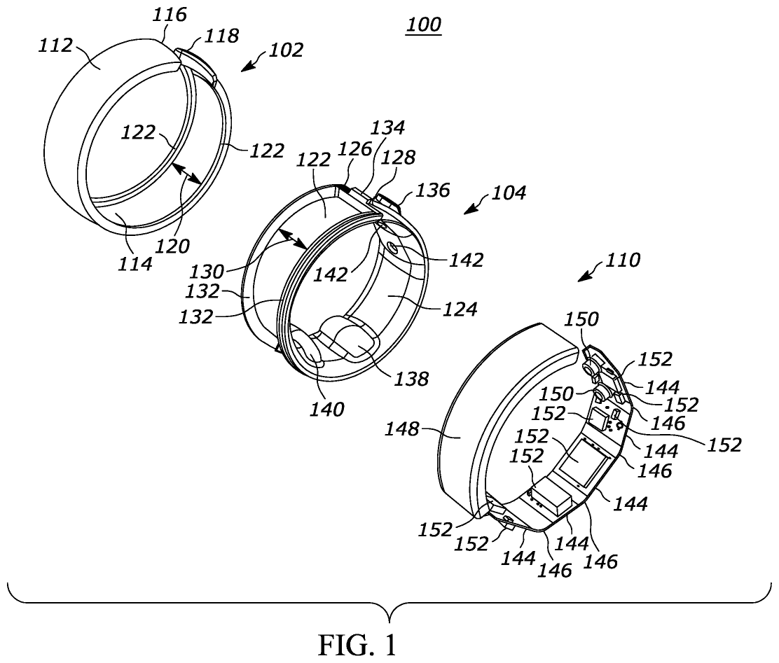

FIG. 1 is an expanded view of an example of a wearable device 100 that includes an outer circular metal piece 102, an inner circular plastic piece 104, and sensor electronics 110 that are enclosed between the outer circular metal piece and the inner circular plastic piece.

The outer circular metal piece 102 includes an outer surface 112, an inner surface 114, and two opposing ends 116 and 118. As used herein, the term “inner surface” of the piece refers to a surface of the piece that is closer (or closest) to the finger on which the device is worn and the term “outer surface” of the piece refers to a surface of the piece that is farther (or farthest) from the finger on which the device is worn. Thus, as shown in FIG. 1, the outer surface 112 of the outer circular metal piece 102 is farther from the finger on which the ring is worn than the inner surface 114 of the outer circular metal piece. In the example of FIG. 1, the outer circular metal piece includes a channel 120 that is formed by the inner surface 114 of the outer circular metal piece. For example, the channel is formed by ridges 122 (also referred to as sidewalls) that run along the outer edges of both sides of the outer circular metal piece. In addition, the two opposing ends 116 and 118 of the outer circular metal piece include attachment features that are shaped to mate with attachment features of the inner circular plastic piece. Examples of the attachment features of the outer circular metal piece are described below.

The inner circular plastic piece 104 includes an outer surface 122, an inner surface 124, and two opposing ends 126 and 128. With reference to FIG. 1, the outer surface 122 of the inner circular plastic piece 104 is further from the finger on which the ring is worn than the inner surface 124 of the inner circular plastic piece. In the example of FIG. 1, the inner circular plastic piece includes a channel 130 that is formed by the outer surface of the inner circular plastic piece. For example, the channel is formed by ridges 132 (also referred to as sidewalls) that run along the outer edges of both sides of the inner circular plastic piece.

The two opposing ends 126 and 128 of the inner circular plastic piece 104 include attachment features 134 and 136 that are shaped to mate with the attachment features of the outer circular metal piece 102. In the example of FIG. 1, the attachment features 134 and 136 of the inner circular plastic piece 104 include protrusions at the opposing ends of the inner circular plastic piece. In an embodiment, the protrusions create ridges that mate with ridges of the attachment features of the outer circular metal piece.

Referring to FIG. 1, the inner circular plastic piece 104 may also include features such as bumps 138 and 140 that are formed to improve performance of the optical sensors. For example, the bumps are formed of transparent plastic that improve optical coupling between the optical electronic components (e.g., the LEDs and the photodetectors) and the skin of a finger on which the ring is worn. For example, a bump that is located next to an LED enables emitted light to couple more efficiently with skin that is pressed against the bump and a bump that is located next to a photodetector enables reflected light to couple more efficiently with the bump that is pressed against the skin. In the embodiment of FIG. 1, the inner circular plastic piece also includes through-holes 142 that enable battery charging contacts to pass through the inner circular plastic piece and contact the skin of a finger on which the ring is worn.

Referring again to FIG. 1, the sensor electronics 110 include a PCB and electronic components. The PCB may be, for example, a flexible PCB or a PCB that includes rigid portions and flexible portions. In the example of FIG. 1, the PCB includes rigid portions 144 and flexible portions 146. Typically, electronic components are attached at the rigid portions of the PCB and the flexible portions of the PCB enable the PCB to be formed into a circular shape. In an embodiment, the electronic components include a curved battery 148, battery charging contacts 150, and various IC devices 152. The IC devices may include, for example, at least one light source (e.g., an LED or an LED bank), a photodetector, a microcontroller, memory, wireless communications (e.g., Bluetooth Low Energy), temperature sensor, RF sensor, motion sensor (e.g., gyroscope), location sensor (e.g., GPS), and/or discrete devices (e.g., capacitors, resistors, and/or inductor). In the example of FIG. 1, some of the visible electronic components include a curved battery, an LED bank, a photodetector, a microcontroller, memory, and battery charging contacts. In an embodiment, one sensor device may include a photodetector IC packaged along with one or more LEDs, e.g., an LED that emits in the IR band and an LED that emits in the red band. In an embodiment, the sensor electronics are configured to monitor activity and/or health parameters of the person wearing the device. For example, the sensor electronics can track activity parameters such as motion and location and health parameters such as heart rate, heart rate variability (HRV), respiration rate, temperature, peripheral oxygen saturation (SpO₂), blood pressure, and/or blood glucose level. Although some examples of sensor parameters are described, other parameters may be monitored. Additionally, the sensor electronics may include electronics configured to implement other functionality such as, for example, electronic payments.

In an embodiment, the outer circular metal piece 102 is formed from metal such as steel, stainless steel, titanium, or aluminum, although other metals are possible. The metal could be a metal alloy and could be, for example, machined (e.g., CNC machined) or molded. In an embodiment, the steel is formed to be somewhat flexible at the corresponding dimensions in order to provide some flexibility in response to typical forces encountered from putting the wearable device on a finger, taking the wearable device off of a finger, and/or in response to changes in the size of a finger (e.g., due to swelling). In an embodiment, the outer circular metal piece is made of spring steel that has a modulus of elasticity that provides a desired level of flexibility.

In an embodiment, the inner circular plastic piece 104 is formed of plastic, including for example, resin, a medical grade polycarbonate, or any other type of synthetic material made from an organic polymer. Typically, the inner circular plastic piece is molded into the desired shape using techniques that are known in the field. Although the inner circular plastic piece is described as being plastic, in other embodiments, the inner circular piece could be formed of some other material.

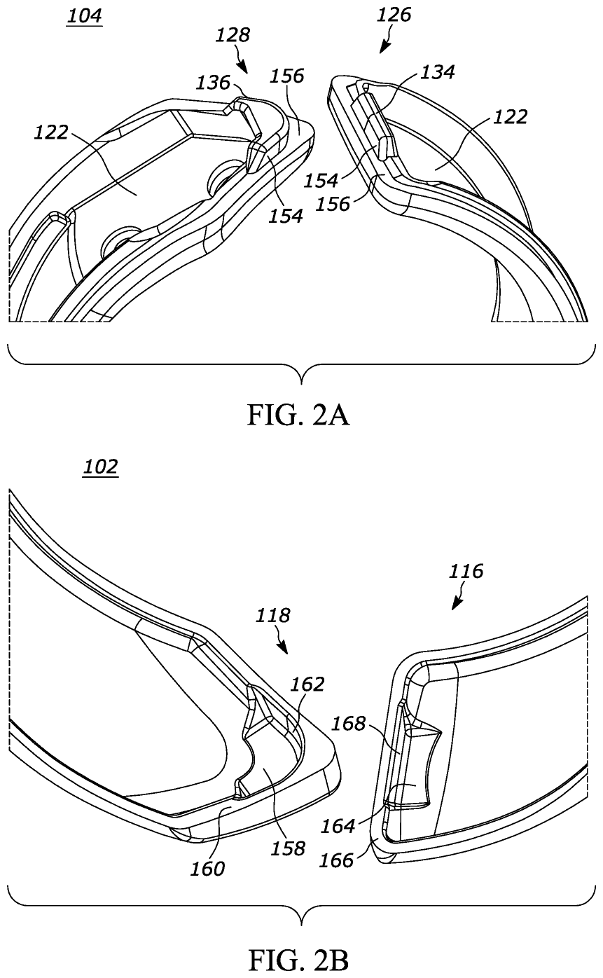

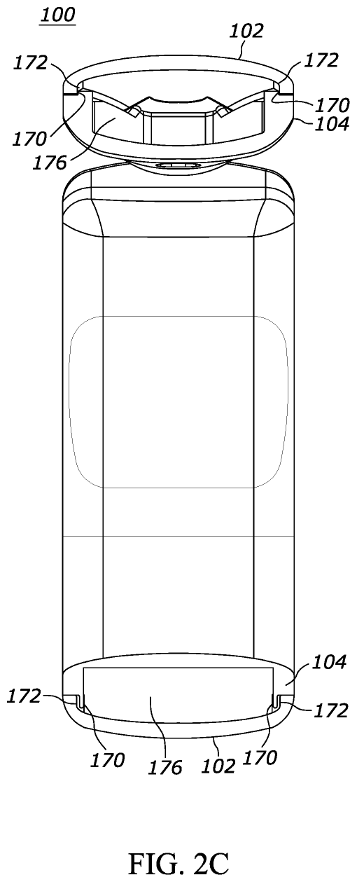

As described above, both the inner circular plastic piece 104 and the outer circular metal piece 102 include attachment features at their opposing ends. FIG. 2A depicts an expanded view of an example of the attachment features 134 and 136 at the two opposing ends of the inner circular plastic piece 104. With reference to the end 128 of the inner circular plastic piece shown on the left side of FIG. 2A, the attachment feature 136 includes a protrusion at the outer surface that creates a ridge 154 that rises above a planar surface 156 of the inner circular plastic piece 104. The protrusion has a particular shape that is complementary to a cavity of the outer circular metal piece 102. With reference to the end 126 of the inner circular plastic piece shown on the right side of FIG. 2A, the attachment feature 134 also includes a protrusion at the outer surface that creates a ridge 154 that rises above a planar surface 156 of the inner circular plastic piece 104. The protrusion has a particular shape that is complementary to a cavity of the outer circular metal piece.

FIG. 2B depicts an example of the attachment features at the two opposing ends 116 and 118 of the outer circular metal piece 102. With regard to the end 118 of the outer circular metal piece 102 shown on the left side of FIG. 2B, the attachment feature includes a cavity 158 at the inner surface that goes below a planar surface 160 of the outer circular metal piece. The cavity has a particular shape that is complementary to the protrusion of the attachment feature 126 of the inner circular plastic piece 104 as shown on the left side of FIG. 2B. For example, the ridge 154 of the protrusion of the inner circular plastic piece mates with a ridge 162 of the cavity 158 of the outer circular metal piece 102. In an embodiment, and as shown in FIGS. 2A and 2B, the shape of the attachment feature of the outer circular metal piece is the inverse of the shape of the attachment feature of the inner circular plastic piece such that the protrusion of the inner circular plastic piece fits snugly into the respective cavity of the outer circular metal piece. With reference to the end 116 of the outer circular metal piece 102 shown on the right side of FIG. 2B, the attachment feature also includes a cavity 164 that goes below a planar surface 166 of the outer circular metal piece 102. The cavity has a particular shape that is complementary to the protrusion of the attachment feature 134 of the inner circular plastic piece 104 as shown on the right side of FIG. 2A. For example, the ridge 154 of the protrusion of the inner circular plastic piece 104 mates with a ridge 168 of the cavity 164 of the outer circular metal piece 102. In an embodiment, and as shown in FIGS. 2A and 2B, the shape of the attachment feature of the outer circular metal piece is the inverse of the shape of the attachment feature of the inner circular plastic piece such that the protrusion of the inner circular plastic piece fits snugly into the respective cavity of the outer circular metal piece.

Although examples of attachment features are shown and described herein, other configurations of attachment features are possible. For example, the attachment features of the inner circular plastic piece 104 may include cavities (e.g., similar to FIG. 2B), while the outer circular metal piece 102 may include protrusions (e.g., similar to FIG. 2A), or some combination thereof.

In other embodiments, complementary attachment features may be located at the sides of the inner circular plastic piece and the outer circular metal piece in addition to, or instead of, the attachment features at the opposing ends of the inner circular plastic piece and the outer circular metal piece. For example, FIG. 2C is a side cutaway view of an assembled ring 100 that shows complementary attachment features located at the sides of the outer circular metal piece 102 and at the sides of the inner circular plastic piece 104. At the top of FIG. 2C, the side cutaway view shows attachment features 170 at the sides of the inner circular plastic piece 104 mated to side ridges 172 of the outer circular metal piece 102. At the bottom of FIG. 2C, the side cutaway view shows attachment features 170 at the sides of the inner circular plastic piece 104 mated to side ridges 172 of the outer circular metal piece 102. In both cases, the inner circular plastic piece 104 includes ridges that mate against ridges of the outer circular metal piece 102. In a fully assembled ring, the cavity 176 between the outer circular metal piece and the inner circular plastic piece is filed with an encapsulant.

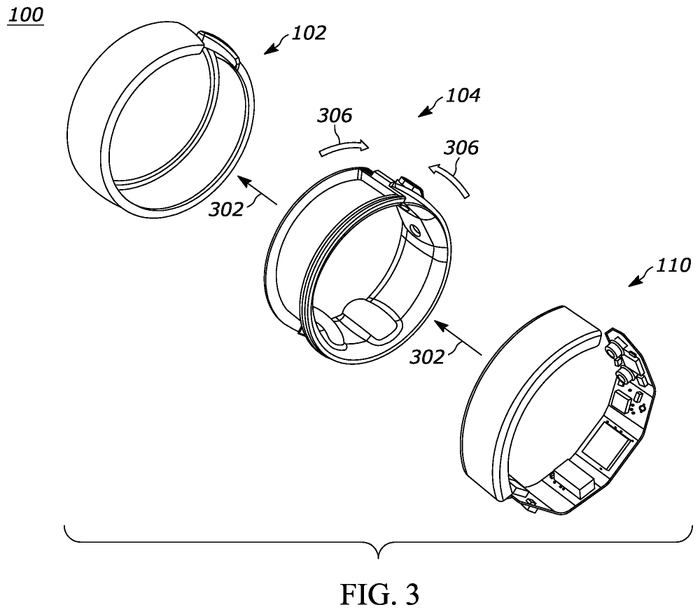

An example assembly process for producing a wearable device is now described with reference to FIG. 3. In a first operation as indicated by arrow 302, the sensor electronics 110 are attached to the inner circular plastic piece 104. For example, the sensor electronics are wrapped around the outer surface of the inner circular plastic piece such that the sensor electronics sit at least partially within the channel that is formed by the outer surface of the inner circular plastic piece. In an embodiment, attaching the sensor electronics to the inner circular plastic piece involves aligning certain electronic components of the sensor electronics with features of the inner circular plastic piece. In the example of FIG. 3, the light source (e.g., an LED bank) is aligned with the bump of the inner circular plastic piece 104, the photodetector is aligned with the bump of the inner circular plastic piece, and the battery charging contacts are aligned with the through holes in the inner circular plastic piece. The attachment process may also involve spot gluing of the sensor electronics to the inner circular plastic piece. In other embodiments, the attachment process may involve aligning physical alignment features (e.g., alignment tabs) of the inner circular plastic piece and the sensor electronics and/or connecting physical connection features of the inner circular plastic piece and/or the sensor electronics.

In an embodiment, the sensor electronics 110 may first be attached to a carrier that includes attachment features that are then attached to complementary attachment features of the inner circular plastic piece. For example, the channel in the inner circular plastic piece may include alignment/attachment features that correspond to alignment/attachment features of a carrier and/or to alignment/attachment features of the sensor electronics. For example, the PCB may include a notch or other alignment feature that corresponds to a protrusion in the channel of the inner circular plastic piece.

Once the sensor electronics 110 are attached to the inner circular plastic piece 104, in a next operation as indicated by arrow 304, the inner circular plastic piece is attached to the outer circular metal piece 102. For example, the inner circular plastic piece is attached to the outer circular metal piece such that the attachment features of the inner circular plastic piece mate with the attachment features of the outer circular metal piece, thereby holding the inner circular plastic piece and the outer circular metal piece securely together. In an embodiment, the inner circular plastic piece is attached to the outer circular metal piece by compressing the inner circular plastic piece so that the inner circular plastic piece is able to snap into place within the outer circular metal piece. For example, compressing the inner circular plastic piece involves applying forces to the inner circular plastic piece that cause the shape of the inner circular plastic piece to change and the two opposing ends of the inner circular plastic piece to move closer to each other, momentarily allowing the inner circular plastic piece to fit within the outer circular metal piece. Forces that cause the two opposing ends of the inner circular plastic piece to move closer to each other are represented by arrows 306 in FIG. 3. Once the inner circular plastic piece is fit within the outer circular metal piece, the forces are removed from the inner circular plastic piece and the opposing ends of the inner circular plastic piece move away from each other to, or nearly to, their position at rest, causing the attachment features of the inner circular plastic piece to mate with the attachment features of the outer circular metal piece. For example, compressing the inner circular plastic piece enables the protrusions at the opposing ends of the inner circular plastic piece to be moved inside of the cavities at the opposing ends of the outer circular metal piece. That is, the protrusions at the opposing ends of the inner circular plastic piece are able to seat within the complementary shaped cavities at the opposing ends of the outer circular metal piece due to the temporarily applied forces that compress the inner circular plastic piece, ultimately resulting in a snug fit between the inner circular plastic piece and the outer circular metal piece. In addition to the mating between the attachment features at the opposing ends of the inner circular plastic piece and the attachment features at the opposing ends of the outer circular metal piece, in an embodiment, the ridges of the inner and outer circular pieces that form the corresponding channels are also configured to touch each other when the inner circular plastic piece and the outer circular metal piece are attached to each other, thereby forming a cavity within which the sensor electronics sit. In an embodiment, the ridges along the circumference of the inner circular plastic piece and the ridges along the circumference of the outer circular metal piece have complementary features (e.g., angular features) that enable the two pieces to fit snuggly together as described with reference to FIG. 2C.

In the example described with reference to FIG. 3, the inner circular plastic piece 104 is compressed to attach the inner circular plastic piece to the outer circular metal piece 102. In another embodiment, the outer circular metal piece may be spread apart (e.g., causing the two opposing ends of the outer circular metal piece to move farther away from each other) so that the inner circular plastic piece can fit within the outer circular metal piece. Once the inner circular plastic piece is fit within the outer circular metal piece, the opposing ends of the outer circular metal piece are allowed to return to, or nearly to, their position at rest. In another embodiment, the attachment process may involve some compressing of the inner circular plastic piece and some spreading of the outer circular metal piece at the same time to enable the attachment features of the two pieces to mate with each other.

In the example described with reference to FIG. 3, the sensor electronics 110 are first attached to the inner circular plastic piece 104. However, in another embodiment, the sensor electronics may first be attached within the channel of the outer circular metal piece and then the inner circular plastic piece is attached to the outer circular metal piece.

After the inner circular plastic piece 104 is attached to the outer circular metal piece 102, in a next operation, the cavity that is formed between the inner circular plastic piece and the outer circular metal piece can be filled with an encapsulant (e.g., a plastic or resin). For example, an encapsulant in a fluid form is injected through a hole in either the inner circular plastic piece or the outer circular metal piece, or through a gap between the inner circular plastic piece and the outer circular metal piece, and allowed to cure within the cavity. The encapsulant may fill in open space in the cavity and harden directly around the electronic components in the cavity and at least partially against the inner surface of the outer circular metal piece and the outer surface of the inner circular plastic piece. Injecting an encapsulant within the cavity can protect the electronic components from damage and can improve the attachment between the inner circular plastic piece and the outer circular metal piece. In an embodiment, the encapsulant is transparent to the wavelengths used for optical sensing.

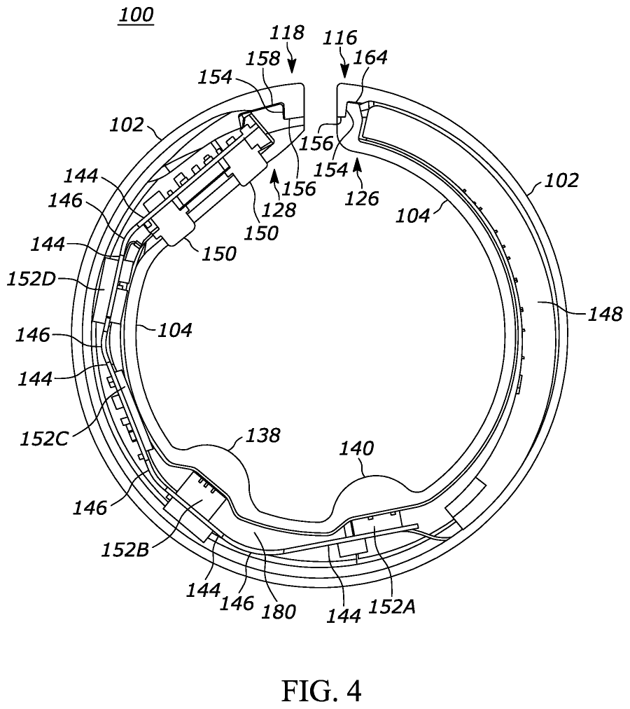

FIG. 4 is a side cutaway view of an example of a fully assembled wearable ring 100, which includes the outer circular metal piece 102, the inner circular plastic piece 104, the sensor electronics 110, and an encapsulant 180 as described with reference to FIGS. 1-3. Elements visible in the side cutaway view include:

Outer circular metal piece 102: opposing ends 116 and 118, cavities 158 and 164.

Inner circular plastic piece 104: LED bump 140, photodetector bump 138, ridges 154, planar surfaces 156.

Sensor electronics: curved battery 148, PCB 144 (rigid) and 146 (flexible), LED 152A, photodetector 152B, battery charging contacts 150, microcontroller 152C, wireless communications 152D.

### Encapsulant 180.

As shown in FIG. 4, the attachment features at the opposing ends 116 and 118 of the outer circular metal piece 102 are mated with the attachment features at the opposing ends 126 and 128 of the inner circular plastic piece 104. As shown, ridges of the attachment features of the outer circular metal piece are engaged with ridges of the attachment features of the inner circular plastic piece such that the two pieces are held snuggly together.

For wearable health monitoring devices, battery life is an important issue and it is desirable to extend the amount of time between battery charges as much as possible. One way to extend the battery life is to use a larger battery, however, this may run counter to the desire to achieve a small form factor, especially for a health monitoring device that is worn on the finger. In the limited space available for the sensor electronics in a finger ring, electronic components such as the photodetector and LEDs are competing for valuable space with the battery.

In addition to the space limitations, it has been found that having multiple LEDs spatially distributed around the ring can provide improved health monitoring. Although it may be desirable to have multiple LEDs spatially distributed around the ring, certain desirable positions of the LEDs may be in conflict with the location of the battery. It has been realized that a specially designed PCB can be utilized to enable sensor components (e.g., an LED bank and/or a photodetector) to be located beneath the battery in a manner that reduces the impact on the overall thickness of the ring while enabling a certain desirable spatial distribution of the sensor components. In an embodiment, such a specially designed PCB includes a first section of the PCB having a first thickness and a second section of the PCB having a second thickness, where the second thickness is less than the first thickness. That is, the second section of the PCB is thinner than the first section of the PCB. With such a specially designed PCB, a curved battery can be positioned over the second section of the PCB and an LED (or LED bank) can be attached to the second section of the PCB opposite the curved battery and positioned under the second section of the PCB such that the LED is separated from the curved battery by the second section of the PCB. Thus, the dual thickness of the PCB enables electronic components to be attached to both sides of the first section of the PCB while also enabling electronic components to be attached to the inner surface of the second section of the PCB opposite the curved battery. For example, a first LED bank can be attached to the inner surface of the first section of the PCB and a photodetector and second LED bank can be attached to the inner surface of the second section of the PCB, which enables the first and second LED banks to be spatially separated from the photodetector at distances that create desirable angles relative to the photodetector while also accommodating a sufficiently large battery.

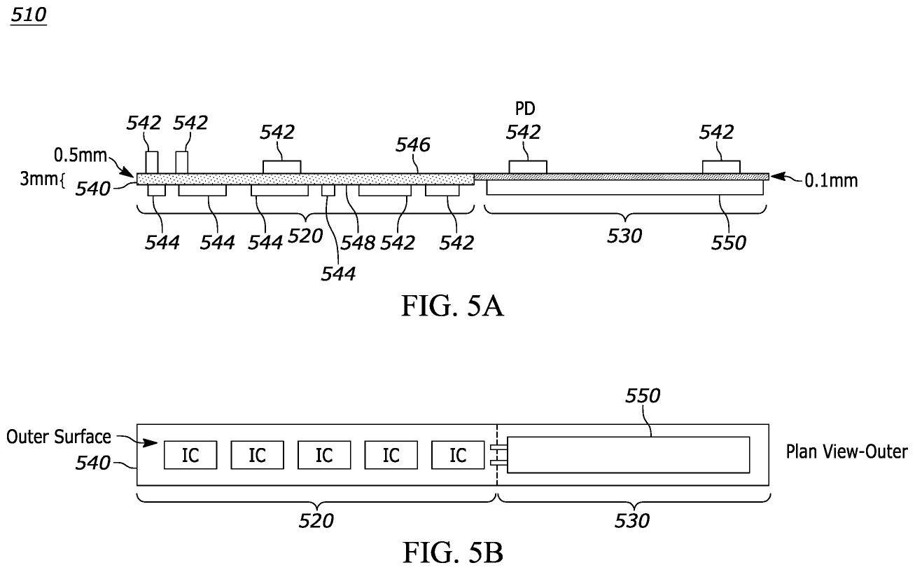

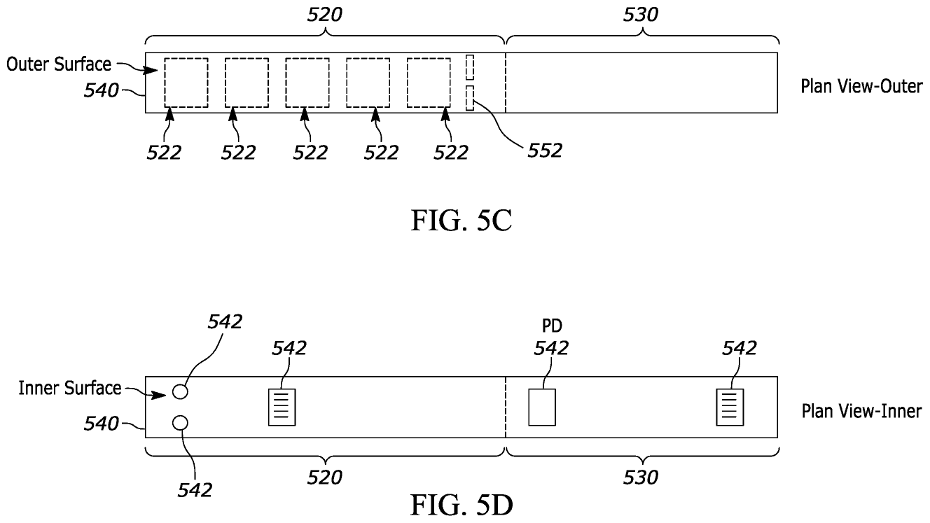

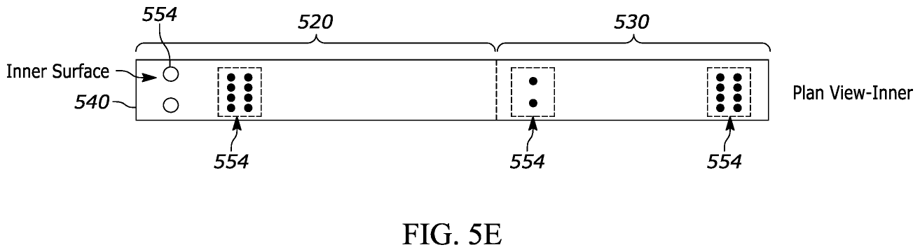

FIGS. 5A-5E depict different views of an example of sensor electronics that includes a PCB having a first section and a second section in which the second section of the PCB is thinner than the first section of the PCB. Note that in FIGS. 5A and 5B the battery is depicted as having no curvature for ease of illustration and description. However, typically, the battery will be curved as shown above in FIGS. 1, 3, and 4.

FIG. 5A is a side view of the sensor electronics 510 that depicts a PCB that includes a first section 520 and a second section 530 in which the second section of the PCB is thinner than the first section of the PCB. In an example embodiment, the first section is about 0.5 mm thick (±10%) and the second section is about 0.1 mm thick (±10%), although other thicknesses are possible. For example, the thickness of the PCB may be a function of how many layers (e.g., metal/conductive layers and insulating layers) that the PCB has. In the example of FIGS. 5A-5E, the inner surface of the PCB is the surface of the PCB that is closest to the finger on which the ring is worn and the outer surface of the PCB is the surface of the PCB that is farthest from the finger on which the ring is worn. As shown in the embodiment of FIG. 5A, the first section of the PCB 540 includes electronic components 542 and 544 attached to both the inner and outer surfaces of the PCB. With reference to FIG. 5A, the inner surface 546 of the first section 520 of the PCB 540 includes an LED bank and battery charging contacts and the outer surface 548 of the first section of the PCB includes various IC devices 544, which may include, for example, a microcontroller, memory, wireless communications, discrete devices (e.g., capacitors and/or resistors), and charging components (e.g., including battery contacts). The inner surface 546 of the second section 530 of the PCB 540 includes a photodetector 542 and an LED bank 542 and the second section 530 of the PCB is between a battery 550 and the photodetector 542 and the LED bank 542 on the second section 530. In an embodiment, the photodetector may include a combined package of LEDs and the photodetector. In the embodiment of FIGS. 5A-5E, the first section of the PCB includes more layers than the second section of the PCB to support electronic components being attached to both the inner surface and the outer surface of the PCB. In an embodiment, the first section 520 of the PCB may include rigid portions (e.g., rigid portions 144, FIG. 1) and flexible portions (e.g., flexible portions 146, FIG. 1). In some embodiments, the rigid portions and the flexible portions of the PCB may have different thicknesses.

FIG. 5B is a plan view of the outer surface 548 of the PCB 540 that shows the various IC devices 544 and the battery 550 (shown here without curvature) relative to the first and second sections 520 and 530 of the PCB 540. To enable attachment of electronic components to the PCB (both physical attachment and electrical connection), the PCB includes component interfaces.

FIG. 5C is a plan view of the outer surface 548 of the PCB 540 without any electronic components attached thereto, which shows an example of various interfaces 552 of the PCB that enable attachment of the electronic components to the PCB. Although not shown in detail, the interfaces of the PCB typically include electrically conductive pads that are exposed at the surface of the PCB to match conductive pads of specific IC devices that are to be attached to the particular interfaces. As is known in the field, IC devices may be attached to the PCB by soldering electrically conductive leads or pads of the IC devices to electrically conductive pads of the PCB. Such soldering can provide physical attachment and electrical connection between the IC devices and the PCB.

FIG. 5D is a plan view of the inner surface 546 of the PCB 540 that shows the battery charging contacts 542, two LED banks 542, and the photodetector 542 relative to the first and second sections 520 and 530 of the PCB. As shown in FIG. 5D, the battery charging contacts and one LED bank are attached at the first section 520 of the PCB and the photodetector and the other LED bank are attached at the second section 530 of the PCB.

FIG. 5E is a plan view of the inner surface 546 of the PCB 540 without any electronic components attached thereto, which shows the interfaces 554 that enable attachment of the electronic components to the PCB. In the example of FIG. 5E, the interfaces include interfaces for the battery charging contacts, the LED banks, and the photodetector.

The PCB design described with reference to FIGS. 5A-5E may also be applicable to a wearable device that is a complete circular ring. That is, the PCB can be included in a ring wearable health monitoring device that is a monolithic circle with no opposing ends.

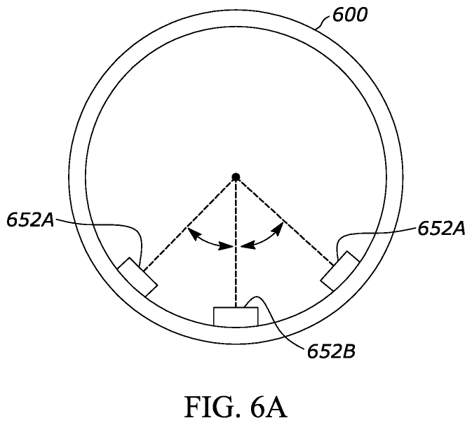

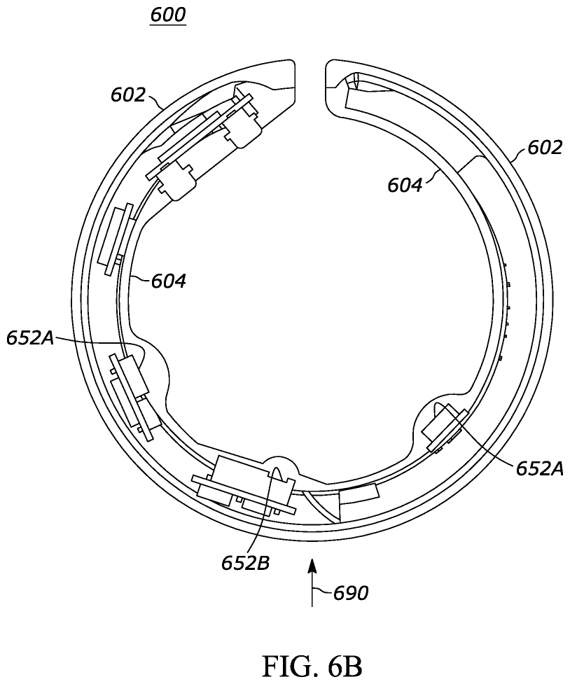

As mentioned above, in some embodiments, the sensor electronics include multiple LEDs spaced around the ring. In one embodiment, the sensor electronics includes two LED banks that are each configured to be at angles of approximately 50 degrees (±20%) relative to the photodetector. FIG. 6A depicts an example angular relationship between two LED banks 652A and a photodetector 652B of a circular wearable health monitoring device 600 in which the LED banks are at approximately 50 degrees (e.g., ±20%) from the photodetector relative to a center point of the ring as illustrated in FIG. 6A.

FIG. 6B is a side cutaway view of an example of a fully assembled wearable ring 600, which includes the outer circular metal piece 602, the inner circular plastic piece 604, and the sensor electronics. The sensor electronics include two LED banks 652A that are spread from the photodetector 652B by approximately 56 degrees (e.g., ±15 degrees or ±5 degrees). In an embodiment, it is desirable that both LED banks have about the same angular spread from the photodetector (e.g., to within ±10 degrees or to within ±5 degrees). In one example, it is desirable that both LED banks have the same angular separation from the photodetector, e.g., to within ±2 degrees).

Because people have different sizes of fingers (e.g., different diameter/circumference), it is desirable to offer rings in different sizes. Although rings may be offered in different sizes, it has been found that certain angular relationships between the light source(s) and the photodetector (e.g., FIGS. 6A and 6B) are desirable across a range of different ring sizes. However, if the same PCB were to be used with two different sized rings (e.g., inner circular plastic piece and outer circular metal piece of different sizes), the angular relationship between the photodetector and the LEDs would be different due to the differences in the diameter of the two different sized rings. In order to maintain a constant angular relationship between the photodetector and the LEDs (e.g., as described with reference to FIGS. 6A and 6B), the linear distance between the LEDs and the photodetector along the PCB would need to be different for different sized rings. Such different spacing would call for unique PCB designs for different sizes of rings. That is, the interfaces for the LEDs and/or photodetector would need to be in different locations on the PCB for each size ring. In order to avoid the need for size-specific layouts of LED interfaces on the PCBs, the PCBs can be fabricated in bulk with multiple interfaces that can be selectively utilized based on the particular size of the ring with which the PCB will be used. For example, PCBs can be manufactured in bulk with multiple interfaces for the LEDs and the particular interfaces that are used can be dependent on the specific size of the particular ring. Thus, the same PCB can be manufactured in bulk and used to support an array of different ring sizes.

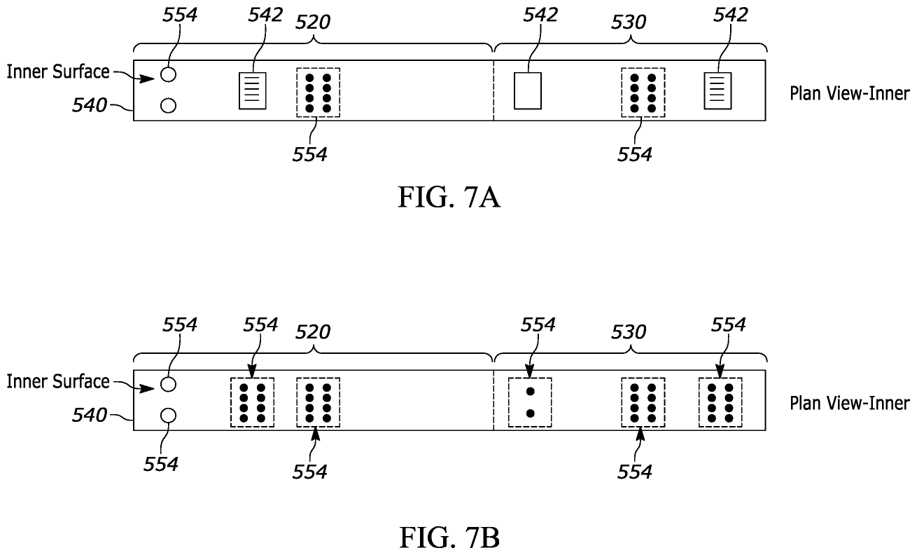

FIGS. 7A and 7B depict an example of the PCB 540 that includes multiple interfaces 544 that can support different positions for LEDs along the length of the PCB. FIG. 7A is a plan view of the inner surface 546 of the PCB that shows the battery charging contacts 542, two LED banks 542, the photodetector 542, and two unused LED bank interfaces 554 relative to the first and second sections 520 and 530 of the PCB. As shown in FIG. 7A, the battery charging contacts and one LED bank are attached at the first section 520 of the PCB and the photodetector and the other LED bank are attached at the second section 530 of the PCB, while one LED bank interface remains unused on each of the first and second sections of the PCB. As shown in FIG. 7A, the PCB includes more LED bank interfaces than LED banks. That is, the PCB (which is to be included within an assembled ring) includes four LED bank interfaces but only two attached LED banks. In such a configuration, the LED bank interfaces that receive the LED banks are dependent on the size of the assembled ring.

FIG. 7B is a plan view of the inner surface 546 of the PCB 540 without any electronic components attached thereto, which shows the interfaces 554 that enable attachment of the electronic components to the PCB. In the example of FIG. 7B, the interfaces include interfaces for the battery charging contacts, the LED banks, and the photodetector. As shown in FIG. 7B, the PCB includes four LED bank interfaces. Although an example with four LED bank interfaces and only two LED banks is described, other configurations of extra LED bank interfaces are possible. Additionally, although the example is described with reference to the LED bank interfaces, the inclusion of extra interfaces on the PCB may be applied to other sensor components whose position on the PCB may change depending on the size of the ring. Additionally, the embodiment of the PCB described with reference to FIGS. 7A and 7B is applicable to an open wearable ring design with opposing ends and to a closed, or monolithic, ring wearable device.

In an embodiment, electronic components, such as an LED bank and/or a photodetector, may be attached to a PCB opposite the battery even with a PCB that does not have dual-thicknesses. For example, in an embodiment, the PCB has the same thickness throughout the length of the PCB, but the combined thickness of the PCB, the battery, and the electronic components attached opposite the battery, are still thin enough to incorporate into a wearable health monitoring device that is worn on the finger.

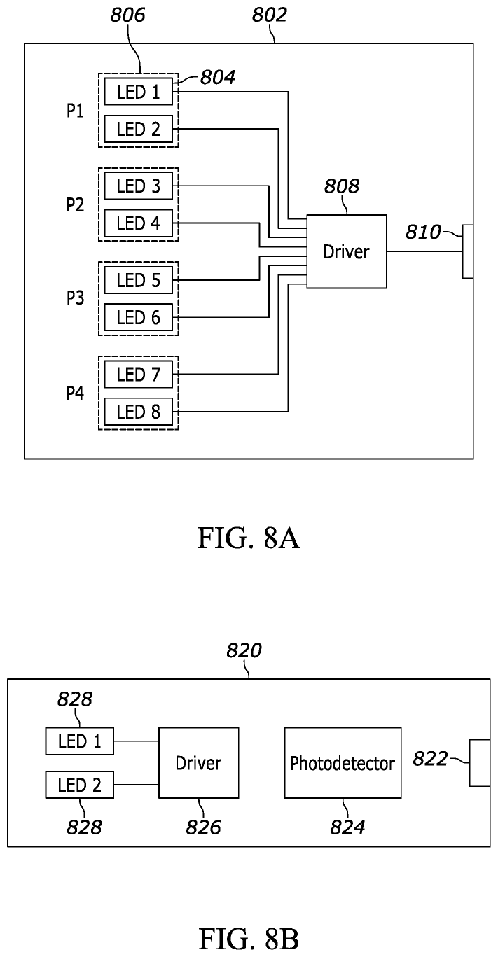

As described above, the light sources may be LED banks that include multiple LEDs integrated together into a single packaged device, referred to herein as an LED bank. FIG. 8A is an example of an LED bank 802 that may be used in a wearable health monitoring device, including a wearable health monitoring device that is intended to be worn on a finger as a ring. In the example of FIG. 8A, the LED bank includes eight different LEDs 804 (LED1, LED2, LED3, LED4, LED5, LED6, LED7, LED8) that are organized into four pairs of LEDs 806 (P1, P2, P3, P4). In the example of FIG. 8A, the LED bank is configured so that each pair of LEDs can be driven individually. In an embodiment, the LED bank has a driver circuit 808 that is configured to drive a particular LED in response to an LED device-specific driver signal. The LED bank also includes an input/output (I/O) port 810 that is configured to receive LED device-specific driver signals to drive the LEDs. In an embodiment, the pairs of LEDs are configured to output different wavelengths of electromagnetic energy. For example, the LEDs may be configured to output electromagnetic energy in the following wavelengths:

$$pair 1 : LED 1 = IR , LED 2 = red ; \\ pair 2 : LED 3 = IR , LED 4 = red ; \\ pair 3 : LED 5 = Far red , LED 6 = orange ; and \\ pair 4 : LED 7 = IR , LED 8 = green .$$

where:

IR=780-1,000 nm, with 840 nm or 940 nm being used in an example;

red=625-740 nm, with 660 nm, 680 nm, or 730 nm (far red) being used in an example;

orange=590-625 nm, with 590 being used in an example;

green=520-565 nm, with 520 nm, 526 nm, or 530 nm being used in an example.

It has been found that different wavelengths of electromagnetic energy may be desirable for monitoring different health parameters. For example, the combination of infrared IR and red in one pair of LEDs may be beneficial for pulse oximetry (e.g., measuring SPO2), while the combination of IR and green in another pair of LEDs may be beneficial for measuring heart rate, heart rate variability, and/or respiratory rate, especially while the device is moving, e.g., due to movement of the wearer of the device. Thus, the activation of the different LED pairs may be tuned to achieve different monitoring goals.

FIG. 8B depicts an example of a combined LED and photodetector packaged into a single device 820. The device includes an I/O port 822, a photodetector 824, a driver 826, and two LEDs 828 (LED1 and LED2). In an embodiment, LED1 is configured to emit electromagnetic energy in the IR band and LED2 is configured to emit electromagnetic energy in the red band. Such a combined photodetector/LED device is known to be used for heart rate and SPO2 monitoring in wearable health monitoring devices.

In an embodiment, a microcontroller of the device is configured to generate only two LED driver signals, referred to herein as “DRV_0” and “DRV_1”. In an embodiment, the LED devices in a wearable device such as that described with reference to FIGS. 6A and 6B that includes two LED banks and a combined LED/photodetector is configured as shown in Table 1.

TABLE 1

| LED Device | LED Wavelength | Driver Signal |
|---|---|---|
| Combined LED/ | LED1 (IR, 940 nm) | DRV_0 |
| Photodetector | LED2 (red, 660 nm) | DRV_1 |
| LED Bank 1/Pair 1 | LED1 (IR, 940 nm) | DRV_0 |
| | LED2 (red, 660 nm) | DRV_1 |
| LED Bank 1/Pair 2 | LED3 (IR, 850 nm) | DRV_0 |
| | LED4 (red, 630 nm) | DRV_1 |
| LED Bank 1/Pair 3 | LED5 (far red, 730 nm) | DRV_0 |
| | LED6 (orange, 590 nm) | DRV_1 |
| LED Bank 1/Pair 4 | LED7 (IR, 850 nm) | DRV_0 |
| | LED8 (green, 526 nm) | DRV_1 |
| LED Bank 2/Pair 1 | LED1 (IR, 940 nm) | DRV_0 |
| | LED2 (red, 660 nm) | DRV_1 |
| LED Bank 2/Pair 2 | LED3 (IR, 850 nm) | DRV_0 |
| | LED4 (red, 630 nm) | DRV_1 |
| LED Bank 2/Pair 3 | LED5 (far red, 730 nm) | DRV_0 |
| | LED6 (orange, 590 nm) | DRV_1 |
| LED Bank 2/Pair 4 | LED7 (IR, 850 nm) | DRV_0 |
| | LED8 (green, 526 nm) | DRV_1 |

In a configuration as shown in Table 1, a single LED driver signal (DRV_0) can be used to simultaneously drive all three of the LEDs configured for IR (940 nm) (e.g., combined LED/photodetector LED1, LED bank 1/Pair 1/LED1, and LED bank 2/Pair 1/LED1) and a single LED driver signal (DRV_1) can be used to simultaneously drive all three of the LEDs configured for red (660 nm) (e.g., combined LED/photodetector LED2, LED bank 1/Pair 1/LED2, and LED bank 2/Pair 1/LED2). For example, it may be desirable to drive both LED banks and the combined LED/photodetector to emit IR (940 nm) and then to emit red (660 nm) to detect heart rate and SPO2. The same set of LED driver signals (DRV_0 and DRV_1) can also be used to drive other pairs of LEDs in the two LED banks. For example, a single LED driver signal (DRV_0) can be used to simultaneously drive both LEDs configured for IR (850 nm) (e.g., LED bank 1/Pair 2/LED3, and LED bank 2/Pair 2/LED3) and a single LED driver signal (DRV_1) can be used to simultaneously drive both LEDs configured for red (630 nm) (e.g., LED bank 1/Pair 2/LED4, and LED bank 2/Pair 2/LED4). Or, a single LED driver signal (DRV_0) can be used to simultaneously drive both LEDs configured for far red (730 nm) (e.g., LED bank 1/Pair 3/LED5, and LED bank 2/Pair 3/LED5) and a single LED driver signal (DRV_1) can be used to simultaneously drive both LEDs configured for orange (590 nm) (e.g., LED bank 1/Pair 3/LED6, and LED bank 2/Pair 3/LED6). Or, a single LED driver signal (DRV_0) can be used to simultaneously drive both LEDs configured for IR (850 nm) (e.g., LED bank 1/Pair 4/LED7, and LED bank 2/Pair 4/LED7) and a single LED driver signal (DRV_1) can be used to simultaneously drive both LEDs configured for green (526 nm) (e.g., LED bank 1/Pair 4/LED8, and LED bank 2/Pair 4/LED8). For example, it may be desirable to drive both LED banks to emit IR (850 nm) and then to emit green (526 nm) to detect heart rate in instances where the device is in motion due to motion of the wearer of the device.

The above-described configuration of LED devices and driver signals can provide a wide variety of wavelengths with which to monitor health parameters of a person while also provided a simple driver signaling configuration.

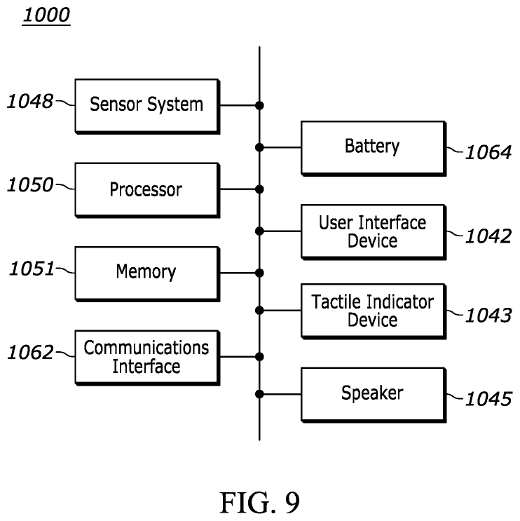

FIG. 9 is an example computing system 1000 that includes a sensor system 1048, a processor 1050, memory 1051, a communications interface 1062, a battery 1064, a user interface device 1042, a tactile indicator device 1043, and a speaker 1045. The computing device may be embodied as a finger wearable device, including, for example, a finger ring as disclosed herein. The computing device may include all of the components or some portion of the components. In an embodiment, the tactile indicator device may include a mechanism that generates tactile feedback (e.g., a vibration) in response to electrical control signal. The sensor system may comprise an optical sensor, an RF-based sensor, and/or some other sensing mechanism. The processor, memory, communications interface, battery, user interface and speaker may be elements as are known in the field.

Since the wearable device is meant to be worn on a finger, the wearable device has dimensions that accommodate human fingers. In an embodiment, the finger wearable device can be produced in sizes that correspond to standard ring sizes of 4-14, which translates to diameters in the range of approximately 20-29 millimeters. The diameter of a circle that aligns with the outer circular metal piece can be in the range of, for example, 20-29 millimeters, the width dimension of the outer circular metal piece may be in the range of, for example, 4.5-10 millimeters, and the thickness of the outer circular metal piece may be in the range of, for example, 0.1-0.7 mm, and preferably in the range of 0.3-0.5 mm.

Referring back to FIGS. 1, 3, and 4, there is a gap, space, or opening between the two ends of the outer circular metal piece and the inner circular plastic piece, e.g., the two opposing ends of the outer circular metal piece and the inner circular plastic piece do not touch each other. As shown in FIGS. 1, 3, and 4, the two ends of the ring are opposite each other but are separated by the gap, space, or opening. Thus, although the outer circular metal piece and the inner circular plastic piece are circular in shape, the two pieces do not form a monolithic circle with no ends and no seams, e.g., as when a metal ring is formed by machining a solid block of metal into a complete ring. Because the ring is not a monolithic circle with no ends and no seams, the finger wearable device is able to have some flexibility. That is, the two ends of the circular ring are able to move relative to each other. In an embodiment, the gap between the two ends of the ring is in the range of, for example, 0.1-10 mm in an at rest state, although other gap sizes are possible. Additionally, although in the examples of FIGS. 1, 3, and 4, the two ends of the ring are not touching each other, in other embodiments, the two ends of the ring may be touching each other. However, even if the two ends of the ring are touching each other, the outer circular metal piece and the inner circular plastic piece do not form a monolithic circle of metal and plastic with no ends and no seams.

The inner circular plastic piece may include one or more raised portions, e.g., portions or “bumps” or “bubbles” that are raised above the inner smooth surface of the inner circular plastic piece. Such raised portions of the encapsulant may be formed to encapsulate larger components of the sensor electronics, to serve as a lens for light from an optical sensor, and/or to provide better contact between the plastic and the skin of the person wearing the device. In an embodiment, the raised portion is aligned with the location of a sensor such as an optical sensor so that the raised portion is directly below the sensor. In an embodiment, as is visible in FIGS. 1 and 3, the raised portion of the inner circular plastic piece spans the width of the inner circular plastic piece and the sensor electronics and is aligned with a sensor, such as an optical sensor, of the sensor electronics. Having a raised portion of the encapsulant that corresponds to the location of an optical sensor and extends across the width of the inner circular plastic piece has been found to promote more consistent contact with the skin and result in improved signal quality from the optical sensor. In an embodiment, an opaque and/or reflective material may be added to the edges of the raised portion to prevent light from a corresponding optical sensor from “leaking” out the sides of the raised portion. For example, some paint (e.g., white paint) may be applied on the edges of the raised portion so that light emitted from the optical sensor is directed through raised portion at the interface between the skin and the raised portion, which can result in improved signal quality from the optical sensor.

As shown in FIGS. 1, 3, and 4, the finger wearable device is not a monolithic circle of metal, plastic, and encapsulant. Because the finger wearable device is not a monolithic circle of metal, plastic, and encapsulant, the finger wearable device is able to have some flexibility, e.g., such that opposing ends of the finger wearable device are able to move or “flex” relative to each other in response to typical forces encountered from putting the wearable device on a finger, taking the wearable device off of a finger, and/or in response to changes in the size of a finger (e.g., due to swelling). The flexibility of the finger wearable device enables a user to wear a tighter fitting device in a typical position below a knuckle than may be possible with a wearable finger ring that has no ability to flex. It has been found that a tighter fitting finger wearable device provides more consistent skin to device contact, which can result in better signal generation from a sensor such as an optical and/or radio frequency (RF) sensor. In some embodiments, the flexibility of the circular metal shell and the flexibility of the encapsulant are matched to each other.

Additionally, it has been found that the finger wearable device will tend to experience the greatest flexing at a point that is opposite the opening. That is, when a spreading force is applied to the ring, e.g., from putting the ring on a finger, taking the ring off a finger, from finger swelling, or from some other externally applied force, the greatest stress and/or strain experienced by the finger wearable device is experienced at the portion of the wearable device that is opposite the opening (e.g., opposite along a line that passes through the opening and through the center point of a circle that is defined by the wearable device). For example, with reference to FIG. 6B, the greatest stress and/or strain may be experienced by the finger wearable device at or near the location indicated by the arrow 690. Given that the finger wearable device will tend to experience the greatest stress and/or strain at a known location, in some embodiments, the sensor electronics are configured so that a more flexible portion of the sensor electronics is aligned with such a location. For example, a flexible portion of the circuit board is positioned at such a location. For example, the sensor electronics are specifically designed and placed within the circular metal shell such that a flexible portion that connects two pieces of circuit board is aligned with the location of greatest stress/strain.

In an embodiment, the inner circular plastic piece and the outer circular metal piece have the general shape of at least a portion of a circle. For example, the inner circular plastic piece and the outer circular metal piece have an identifiable center and radius.

In the embodiments described with reference to FIGS. 1, 3, and 4, the wearable device has a “planar” configuration in which a center of a circle that corresponds to the outer circular metal piece and a center line of the outer circular metal piece are coplanar. That is, a plane could be fit to intersect the centerline of the outer circular metal piece and the center of a circle that is at least partially formed by the outer circular metal piece or the finger wearable device could lay flat against a flat surface with an entire side edge of the wearable device in contact with the flat surface when the device is resting on the flat surface. However, in other embodiments, the wearable device may have a “spiral” shape. With a spiral shape, the entire side of the wearable device is not in contact with a flat surface when the finger wearable device is at rest on its side on the flat surface. Other configurations of the finger wearable device are also possible.

As described above with regard to FIG. 1, after the inner circular plastic piece 104 is attached to the outer circular metal piece 102, the cavity that is formed between the inner circular plastic piece and the outer circular metal piece can be filled with an encapsulant (e.g., a plastic or resin). For example, an encapsulant in a fluid form is injected through a hole in either the inner circular plastic piece or the outer circular metal piece, or through a gap between the inner circular plastic piece and the outer circular metal piece, and allowed to cure within the cavity. The encapsulant may fill in open space in the cavity and harden directly around the electronic components in the cavity and at least partially against the inner surface of the outer circular metal piece and the outer surface of the inner circular plastic piece. However, it has been found that due to the snug fit between the inner circular plastic piece and the outer circular metal piece, the injection of the encapsulant through a hole can trap gas withing the cavity, which may cause pressure to build up within the cavity preventing the entire cavity from being filled with encapsulant. Thus, it has been realized that an exhaust hole may be provided in the cavity so that gas will be exhausted from the cavity as the cavity is filed with encapsulant. Further, the locations of the two holes, referred to as an injection hole and an exhaust hole, are important to ensure that the cavity is able to be completely filed with encapsulant. In particular, it has been realized that locating the injection hole and the exhaust hole at opposing ends of the cavity enables gas in the cavity to be fully exhausted as the cavity is filled with encapsulant. Thus, in an example embodiment, the injection hole and the exhaust hole are located at opposing ends of the inner circular plastic piece. Additionally, in order to preserve a smooth surface at the interface of the skin, the injection hole and the exhaust hole are located on a sidewall of the inner circular plastic piece in an area that is not typically in contact with the skin of the person while the ring is worn on a finger.

In an example embodiment, the inner circular plastic piece includes an injection hole and an exhaust hole. The injection hole is configured to enable encapsulant to be injected into a cavity that is created between the inner circular plastic piece and the outer circular metal piece when the inner circular plastic piece is connected to the outer circular metal piece. The exhaust hole is configured to enable gas to escape the cavity as the cavity is being filled with the encapsulant from the injection hole. In an embodiment, both the injection hole and the exhaust hole are located on a sidewall of the inner circular plastic piece, which helps to maintain a smooth inner surface of the plastic piece, e.g., the surface that is in direct contact with the finger while being worn. In an embodiment, the injection hole and the exhaust hole are located at opposing ends of the inner circular plastic piece, for example, the injection hole is located at a first (e.g., flat) end of the inner plastic piece and the exhaust hole is located at a second (e.g., pointed) end of the inner plastic piece. In other embodiments, the injection hole is located at the second end of the inner plastic piece and the exhaust hole is located at the first end of the inner plastic piece that is opposite the first end. In an embodiment, the injection hole and the exhaust hole are located at opposing ends of the inner circular plastic piece in order to enable gas to be exhausted from the cavity until the entire cavity is filed with encapsulant. If the two holes were located closer to each other, it may cause the exhaust hole to become plugged with encapsulant before the entire cavity is able to be filed with the encapsulant. Additionally, if an exhaust hole were not provided, pressure may build up within the cavity during injection of the encapsulant, which may prevent further filling of the cavity with encapsulant and/or cause the outer metal piece to separate from the inner metal plastic after the inner circular plastic piece has been connected to the outer circular metal piece.

In an embodiment, filling the cavity with encapsulant involves drawing a vacuum through the exhaust hole while injecting encapsulant through the injection hole. It has been found that creating a vacuum within the cavity while encapsulant is being injected into the cavity can help to more thoroughly fill the cavity with encapsulant and/or to more quickly fill the cavity with encapsulant.

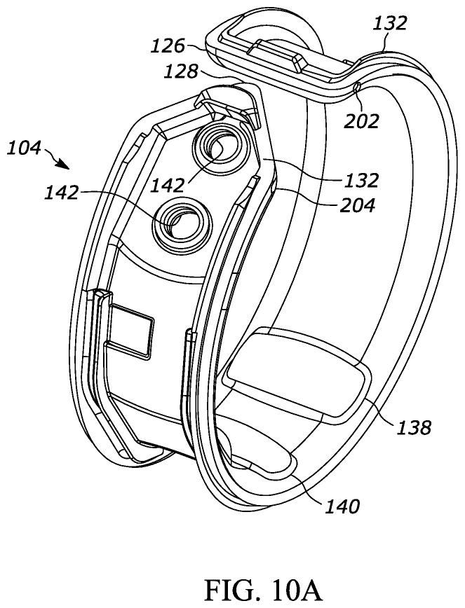

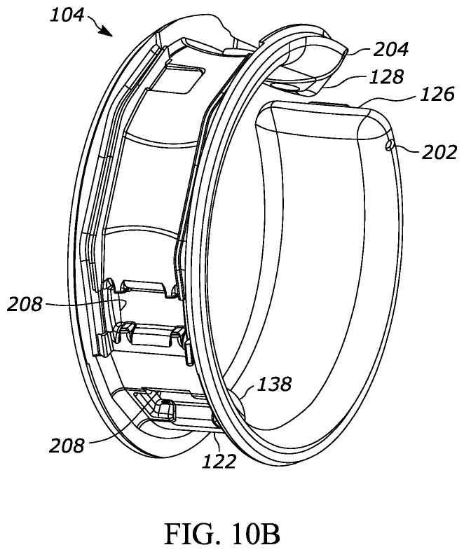

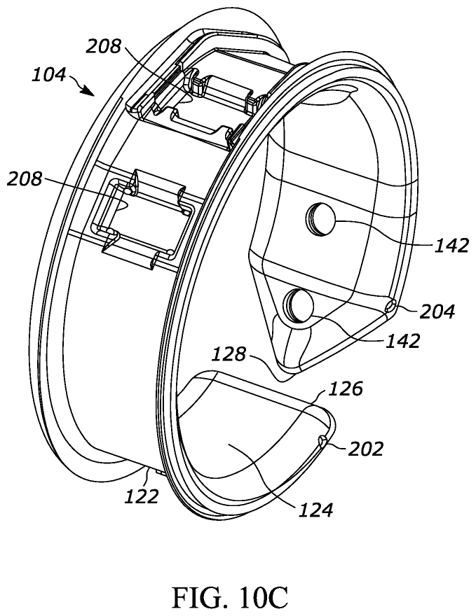

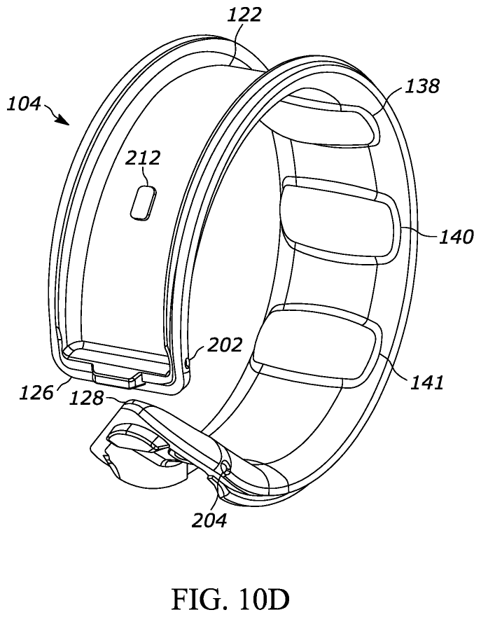

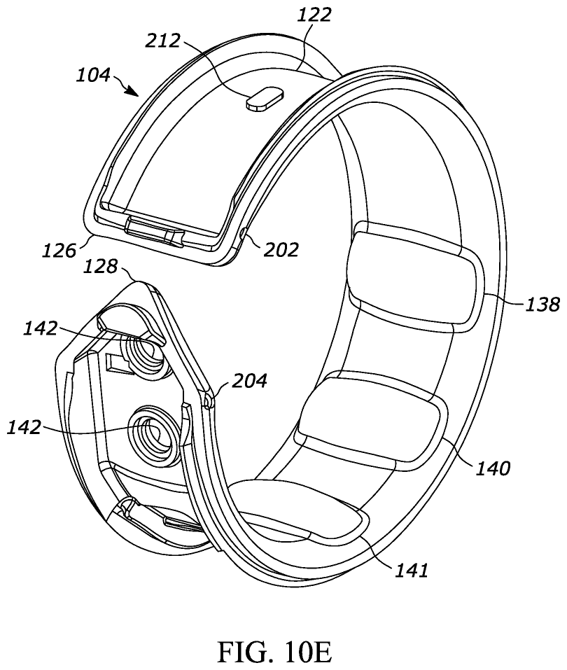

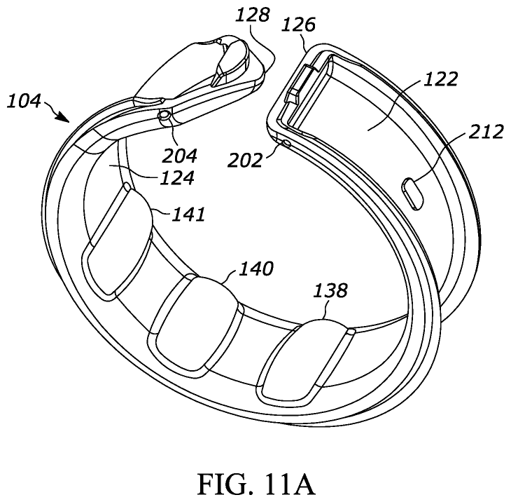

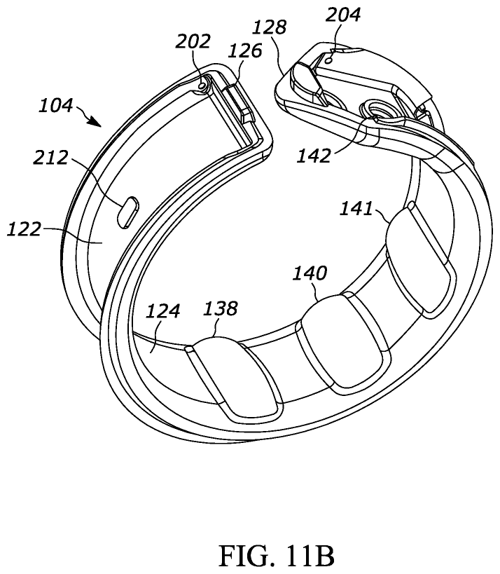

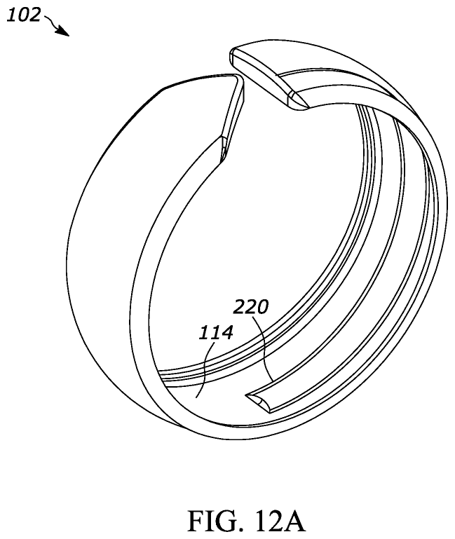

Examples of the inner circular plastic piece are now described in more detail with reference to FIGS. 10A-10E, 11A, and 11B.

FIG. 10A is a perspective view of an example of the inner circular plastic piece 104. FIG. 10A includes a view of an injection hole 202 on a sidewall portion (ridge 132) of the inner circular plastic piece. In the example shown in FIG. 10A, the injection hole is located near the flat end 126 (first opposing end) of the inner circular metal piece. In FIG. 10A, an exhaust hole 204 is also partially shown at the pointed end 128 (second opposing end) of the inner circular metal piece on a sidewall portion (ridge 132) of the inner circular plastic piece. FIG. 10A also includes the through-holes 142 that are configured to receive the battery charging contacts (FIG. 1, 150) and bumps 138 and 140.

FIG. 10B is another perspective view of the inner circular plastic piece 104, which is similar to the perspective view of FIG. 10A except that the inner circular plastic piece is rotated in the clockwise direction compared to the view of FIG. 10A. FIG. 10B includes a view of the injection hole 202 on the sidewall portion of the inner circular plastic piece. In the example shown in FIG. 10B, the injection hole is located near the flat end 126 (first opposing end) of the inner circular metal piece. FIG. 10B also includes a view of the exhaust hole 204 on the sidewall portion of the inner circular plastic piece. In the example shown in FIG. 10B, the exhaust hole is located near the pointed end 128 (second opposing end) of the inner circular metal piece.

FIG. 10B also includes two component bays 208 that are configured to receive electronic sensor components that are part of the sensor electronics (FIG. 1, 110). In the example of FIG. 10B, the two component bays are configured to receive optical components (e.g., a light source, a photodetector, or a combination light source and photodetector), although the component bays may be configured to receive other electronic components. In the embodiment of FIG. 10B, the component bays are generally rectangular in shape and have a depth of about 2-10 mm below a planar surface of the outer surface 122 of the inner circular plastic piece 104, although other depths are possible. In an example embodiment, the component bays are sized to closely match the size of the component that will be received. For example, the footprint dimensions of the components bays exceed footprint dimensions of the corresponding components by less than about 2 mm, or in other embodiments, less than 1 mm. A snug fit of a component in a component bay can help hold the sensor electronics in place on the inner circular plastic piece without any adhesive and before the inner circular plastic piece plus sensor electronics are attached to the outer circular metal piece. The component bays may be aligned with the bumps on the inner circular plastic piece. Although two component bays are shown, different numbers of component bays are possible. Additionally, the size and/or shape of each component bay may be different from those shown in FIG. 10B.

FIG. 10C is another perspective view of the inner circular plastic piece 104. The perspective view of FIG. 10C is similar to the perspective view of FIG. 10B except that the inner circular plastic piece is rotated in the clockwise direction compared to the view of FIG. 10B. FIG. 10C includes a view of the injection hole 202 on the sidewall portion of the inner circular plastic piece. In the example shown in FIG. 10C, the injection hole is located near the flat end 126 (first opposing end) of the inner circular metal piece. FIG. 10C also includes a view of the exhaust hole 204 on the sidewall portion of the inner circular plastic piece. In the example shown in FIG. 10C, the exhaust hole is located near the pointed end 128 (second opposing end) of the inner circular metal piece.

FIG. 10C also includes views of the same two component bays 208 as shown in FIG. 10B. As shown in FIG. 10C, the component bays are generally rectangular in shape and have a depth of about 2-10 mm below the outer surface 122 of the inner circular plastic piece 104. The component bays may be aligned with the bumps (not visible) on the inner surface 124 of the inner circular plastic piece. FIG. 10C also shows the through-holes 142, which are configured to receive the battery charging contacts (FIG. 1, 150), from the opposite side as in FIG. 10A.

FIG. 10D is another perspective view of the inner circular plastic piece 104. The perspective view of FIG. 10D is similar to the perspective view of FIG. 10C except that the inner circular plastic piece is rotated in the clockwise direction compared to the view of FIG. 10C. FIG. 10D includes a view of the injection hole 202 on the sidewall portion of the inner circular plastic piece near the flat end 126 (first opposing end) of the inner circular metal piece, and a view of the exhaust hole 204 on the sidewall portion of the inner circular plastic piece near the pointed end 128 (second opposing end) of the inner circular metal piece.

Additionally, in the view of FIG. 10D, the two component bays 208 are no longer visible on the outer surface of the inner circular plastic piece although bumps 138, 140, and 141 are now visible on the inner surface of the inner circular plastic piece. In an example embodiment, two of the bumps align with the two component bays visible in FIGS. 10B and 10C.

FIG. 10D also includes a view of an alignment feature 212 on the outer surface 122 of the inner circular plastic piece 104. In the embodiment of FIG. 10D, the alignment feature is a protrusion or bump on the outer surface of the inner circular plastic piece that extends above a plane of the outer surface of the inner circular plastic piece. For example, the protrusion is a rectangular protrusion that extends about 1-5 mm above the plane of the outer surface of the inner circular plastic piece. In the example embodiment, the alignment feature is configured to mate with a corresponding alignment feature of the sensor electronics. For example, the rectangular protrusion on the outer surface of the inner circular plastic piece corresponds to a similarly sized and shaped recess in a portion of the sensor electronics, e.g., a recess in a portion of the PCB of the sensor electronics.

FIG. 10E is another perspective view of the inner circular plastic piece 104. The perspective view of FIG. 10E is similar to the perspective view of FIG. 10D except that the inner circular plastic piece is rotated in the clockwise direction compared to the view of FIG. 10D.

In the view of FIG. 10E, the injection hole 202 is visible on the sidewall portion of the inner circular plastic piece near the flat end 126 (first opposing end) of the inner circular metal piece, and the exhaust hole 204 is visible on the sidewall portion of the inner circular plastic piece near the pointed end 128 (second opposing end) of the inner circular metal piece. Additionally, the two through holes 142 and the alignment feature 212 are visible on the outer surface 122 of the inner circular plastic piece and the three bumps 138, 140, and 141 are visible on the inner surface of the inner circular plastic piece. FIG. 10E also shows that the alignment feature protrudes above the plane of the outer surface of the inner circular plastic piece.

FIG. 11A is another perspective view of the inner circular plastic piece 104. FIG. 11A includes a view of the injection hole 202 and the exhaust hole 204 on the sidewall portion of the inner circular plastic piece. As shown in FIG. 11A, the injection hole is located near the flat end 126 (first opposing end) of the inner circular metal piece and the exhaust hole is located near the pointed end 128 (second opposing end) of the inner circular metal piece. FIG. 11A also shows the three bumps 138, 140, and 141 on the inner surface 124 and the alignment feature 212 on the outer surface 122 of the inner circular plastic metal piece.

FIG. 11B is another perspective view of the inner circular plastic piece 104 from the opposite side of the perspective view of FIG. 11A. FIG. 11B includes a view of the injection hole 202 and the exhaust hole 204 from the opposite side of the sidewall portion of the inner circular plastic piece, which shows that the injection hole and the exhaust hole pass completely through the sidewall (ridge 132) of the inner circular plastic piece. As shown in FIG. 11B, the injection hole is located near the flat end 126 (first opposing end) of the inner circular metal piece and the exhaust hole is located near the pointed end 128 (second opposing end) of the inner circular metal piece. FIG. 11B also shows the three bumps 138, 140, and 141 on the inner surface 124 of the inner circular plastic piece and the alignment feature 212 and a partial view of the through-holes 142 on the outer surface 122 of the inner circular plastic metal piece.

As shown in FIGS. 10A-10E, 11A, and 11B, the injection hole 202 and the exhaust hole 204 are located at opposing ends of the inner circular plastic piece 104 such that when the inner circular plastic piece is connected to the outer circular metal piece (FIG. 1, 102), the injection hole and the exhaust hole are located at opposite ends of the cavity that is formed between the inner circular plastic piece and the outer circular metal piece. As described with reference to FIGS. 10A-10E, 11A, and 11B, the injection hole and the exhaust hole are at opposing ends of the inner circular plastic piece. In example embodiments, the injection hole and exhaust hole are at the ends of the inner circular plastic piece when the holes are located within 10% of the total circumferential length of the inner circular plastic piece to the respective end. In other example embodiments, the injection hole and exhaust hole are at the ends of the inner circular plastic piece when the holes are located closer to the respective ends, e.g., within 5% of the total circumferential length of the inner circular plastic piece to the respective end.

In other examples, the injection hole 202 and/or the exhaust hole 204 may be located in the outer circular metal piece. In still other examples, the injection hole and/or the exhaust hole may be formed at the interface between the inner circular plastic piece and the outer circular metal piece, by for example, grooves in a sidewall of the inner circular plastic piece and/or in the outer circular metal piece. In still other example embodiments, the injection hole and the exhaust hole may be located in opposing sidewalls of the inner circular plastic piece and/or the outer circular metal piece. In still other example embodiments, the injection hole may be located in the inner circular plastic piece and the exhaust hole may be located in the outer circular metal piece, or vice versa. In an example embodiment, the injection and exhaust holes have a diameter in the range of 0.25-2 mm, although other diameters are possible.

Because the wearable device is to be worn on a finger, there is a desire to avoid the wearable device being so thick that it is uncomfortable to wear. However, if elements of the wearable device are too thin, then the structural integrity of the wearable device may be compromised. For example, the structural integrity of the ring may be compromised, especially around the location of the battery. Additionally, it has been found that the integrity of the battery can be jeopardized if the battery is subjected to forces that cause the battery to flex while installed within the ring. Thus, it has been realized that a structural support element could be added to the outer circular metal piece (FIG. 1, 102) to improve the structural integrity of the outer circular metal piece. The structural support element can stiffen the outer circular metal piece at the location of the structural support element. Thus, a structural support element that is integrated with the outer circular metal piece at a location that corresponds to the location of the battery can help to limit the battery from being subjected to forces that may cause the battery to flex.

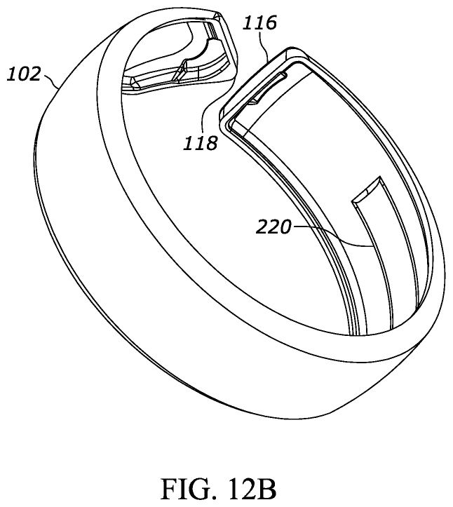

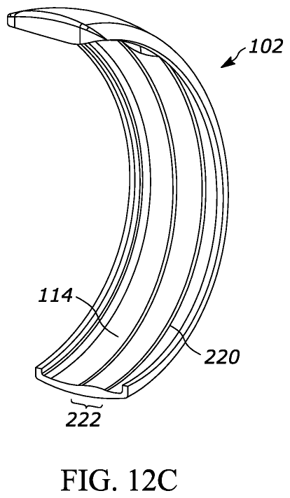

FIG. 12A is a perspective view of the outer circular metal piece 102 that includes a structural support element 220 that is configured to improve the structural integrity of the outer circular metal piece. In the example of FIG. 12A, the structural support element is a stiffening rib that is located at the inner surface 114 of the outer circular metal piece. For example, the stiffening rib is a strip of metal with increased metal thickness along the centerline of the outer circular metal piece. In an embodiment, the stiffening rib is located in a position that coincides with the position of the battery (FIG. 1, 148) in an assembled wearable device, e.g., finger ring. In the view of FIG. 12A, the stiffening rib extends to about the middle of the outer circular metal piece (e.g., the center of the circumferential length of the outer circular metal piece). In an example embodiment, the end of the stiffening rib shown in FIG. 12A is about half way between the flat end 116 and the pointed end 118 of the outer circular metal piece and coincides with an end of the battery in an assembled wearable device.

FIG. 12B is another perspective view of the outer circular metal piece 102 that shows the opposite end of the structural support element 220, e.g., the stiffening rib. In the view of FIG. 12B, the stiffening rib extends towards the flat end 116 of the outer circular metal piece. In an example embodiment, the end of the stiffening rib shown in FIG. 12B coincides with the opposing end of the battery in an assembled ring.

FIG. 12C is a cross section view of the outer circular metal piece 102 that includes the structural support element 220, e.g., the stiffening rib. As shown in FIG. 12C, the stiffening rib is a section of increased thickness of the outer circular metal piece that has a curved shape. The section of increased thickness 222 along the inner surface 114 of the outer circular metal piece improves the structural integrity of the outer circular metal piece, particularly around the areas that coincide with the battery. In an example embodiment, the stiffening rib, has a curved surface that, at its thickest point, increases the thickness of the inner circular metal piece by around 50%, and in another example, the stiffening rib increases the thickness of the inner circular metal piece by around 20% (+5%). In one example embodiment, the outer circular metal piece has a thickness of around 2.5 mm (+10%) and a thickness of 2.72 mm (+10%) at the thickest point of the stiffening rib Although a particular shape and size of the structural support element is described with reference to FIGS. 12A-12C, other shapes and sizes of the structure support element are possible. Additionally, although the example structural support element is an area of increased thickness of the metal of the outer circular metal piece, in other embodiments, the structural support element may be an element that is bonded to the outer circular metal piece. For example, the structural support element may be a separate strip of metal that is attached (e.g., glued or welded) to the inner surface 114 of the outer circular metal piece. For example, the structural support element may be a strip of material that is stiffer than the metal of the outer circular metal piece, such that the outer circular metal piece is stiffer at the location of the structural support element.

Additional disclosure is provided below.

A finger wearable device for health monitoring includes an outer circular piece, the outer circular piece having two opposing ends, an inner circular piece, the inner circular piece having two opposing ends, sensor electronics including a PCB and electronic components connected to the PCB, the sensor electronics encapsulated in a cavity between the outer circular piece and the inner circular piece, wherein the outer circular piece and the inner circular piece include attachment features that are complementary to each other and configured to enable the outer circular piece and the inner circular piece to mate together at the attachment features such that the outer circular piece and the inner circular piece are held together by the attachment features and such that the outer circular piece and the inner circular piece create the cavity within which the sensor electronics are encapsulated, and an encapsulant in the cavity between the outer circular piece and the inner circular piece.

In an example, the attachment features of the inner circular piece are located at the two opposing ends of the inner circular piece, and the attachment features of the outer circular piece are located at the two opposing ends of the outer circular piece.

In an example, the attachment features of the inner circular piece are located at the two opposing ends of the inner circular piece, the attachment features of the outer circular piece are located at the two opposing ends of the outer circular piece, and the attachment features at the two opposing ends of the inner circular piece include protrusions that create ridges.

In an example, the attachment features of the inner circular piece are located at the two opposing ends of the inner circular piece, the attachment features of the outer circular piece are located at the two opposing ends of the outer circular piece, the attachment features at the two opposing ends of the inner circular piece include protrusions that create ridges, and the attachment features at the two opposing ends of the outer circular piece include cavities to receive the ridges of the protrusions of the inner circular piece.

In an example, the attachment features include attachment features at the two opposing ends of the outer circular piece and attachment features at the two opposing ends of the inner circular piece.

In an example, the attachment features of the inner circular piece include protrusions that create ridges at the two opposing ends of the inner circular piece, and the attachment features of the outer circular piece include surfaces at the two opposing ends of the outer circular piece that mate with the ridges at the opposing ends of the inner circular piece.

In an example, the inner circular piece includes an inner surface and an outer surface and a channel formed by the outer surface, wherein the sensor electronics are attached within the channel formed by the outer surface.

In an example, the encapsulant is injected into the cavity after the outer circular piece and the inner circular piece are mated together.

In an example, the encapsulant is injected into the cavity through a hole in the inner circular piece.

In an example, the encapsulant is injected into the cavity through a hole in the outer circular piece.

In an example, the encapsulant is injected into the cavity through a gap between the inner circular piece and the outer circular piece.

In an example, the device further includes a first hole in the inner circular plastic piece and a second hole in the inner circular piece.

In an example, the first hole is located at a first one of the two opposing ends of the inner circular piece and the second hole is at a second one of the opposing ends of the inner circular piece.

In an example, the device further including an injection hole in the inner circular piece and an exhaust hole in the inner circular piece.

In an example, the injection hole is located at a first one of the two opposing ends of the inner circular piece and the exhaust hole is at a second one of the opposing ends of the inner circular plastic piece.

In an example, the encapsulant is injected into the cavity through the injection hole and gas is exhausted through the exhaust hole.

In an example, the injection hole and the exhaust hole are located in a sidewall of the inner circular piece.

In an example, the electronic components include a curved battery, a photodetector, and two LED banks electrically connected to the PCB, wherein the photodetector is attached to the PCB between the two LED banks and a first one of the two LED banks is separated from the curved battery by the PCB.

In an example, the PCB has a first section and a second section in which the first section is thicker than the second section, and wherein the first one of the two LED banks is attached to the PCB at the second section.

In an example, the PCB includes a first section and a second section, wherein the second section is thinner than the first section and wherein the first section has electronic components attached on both an inner side and an outer side of the PCB and the second section has electronic components attached on only an inner side of the PCB.

In an example, the electronic components include a curved battery, a photodetector, and two LED banks connected to the PCB, wherein the photodetector is attached to the PCB between the two LED banks and one of the two LED banks is attached at an inner surface of the PCB directly opposite from the curved battery and separated from the curved battery by the PCB.

In an example, the electronic components include two LED banks and a photodetector connected to the PCB, wherein the photodetector is between the two LED banks on the PCB.

In an example, the electronic components include two LED banks and a photodetector connected to the PCB, wherein the photodetector is between the two LED banks on the PCB, and wherein the two LED banks are located on the PCB at an angle of approximately 50 degrees from the photodetector.

In an example, the inner circular piece has two through holes that pass from the inner surface to the outer surface, and wherein battery charging contacts are located within the two through holes, the battery charging contacts being electrically connected to a curved battery in the cavity.

In an example, the inner circular piece has ridges that form a channel at an outer surface of the inner circular piece, and the outer circular piece has ridges that from a channel at an inner surface of the outer circular piece, wherein the ridges of the inner circular piece are mated with the ridges of the outer circular piece to create the cavity.

In an example, the ridges of the inner circular piece are mated with the ridges of the outer circular piece around a circumference of the inner circular piece and a circumference of the outer circular piece.

In an example, the electronic components include two LED banks and a photodetector connected to the PCB, wherein each LED bank includes at least two pairs of LEDs that are driven together as a pair.

In an example, the electronic components include two LED banks and a photodetector connected to the PCB, wherein each LED bank includes at least two pairs of LEDs that are driven together as a pair, wherein the at least two pairs of LEDs includes a first pair of an infrared (IR) LED and a red LED, and a second pair of an IR LED and a green LED.

In an example, the inner circular piece includes a component bay to receive an electronic component of the sensor electronics.

In an example, the component bay includes a recess below a plane of an outer surface of the inner circular piece.

In an example, the inner circular piece includes an alignment feature on an outer surface of the inner circular piece.

In an example, the alignment feature is a protrusion on the outer surface of the inner circular piece.

In an example, the inner circular piece is an inner circular plastic piece that includes a component bay to receive an electronic component of the sensor electronics.

In an example, the inner circular plastic piece includes an alignment feature on an outer surface of the inner circular plastic piece.

In an example, the outer circular piece includes a structural support element at an inner surface of the outer circular piece.

In an example, the outer circular piece includes a stiffening rib at an inner surface of the outer circular piece.

In an example, the stiffening rib is an area of increased thickness of the outer circular piece.

In an example, the outer circular piece includes a stiffening rib at an inner surface of the outer circular piece, wherein the stiffening rib is an area of increased thickness of the outer circular piece, and wherein the stiffening rib coincides with a location of a curved battery of the sensor electronics.

An apparatus for a finger wearable device for health monitoring is also disclosed. The apparatus includes a PCB having an outer surface and an inner surface, electronic components connected to the PCB, the electronic components including a first LED bank, a second LED bank, a photodetector, and a curved battery, wherein the PCB includes a first section having a first thickness and a second section having a second thickness, the second thickness being less than the first thickness, wherein the first LED bank is connected to the first section of the PCB at the inner surface, the second LED bank is connected to the second section of the PCB at the inner surface, and the photodetector is connected to the PCB at the inner surface between the first LED bank and the second LED bank, and wherein the second LED bank is separated from the curved battery by the second section of the PCB.

In an example, The apparatus of claim 39, wherein the first section of the PCB includes more layers than the second section of the PCB.

In an example, the PCB includes at least three LED bank interfaces.

In an example, the PCB includes at least three LED bank interfaces but only two LED banks are attached to the PCB.

In an example, the PCB includes more LED bank interfaces than LED banks attached thereto.

In an example, the PCB includes more than one LED bank interface on at least one of the first section and the second section of the PCB.

In an example, the photodetector is connected to the second section of the PCB at the inner surface and the curved battery is directly adjacent to the second section of the PCB, and wherein the photodetector and the second LED bank are separated from the curved battery by the second section of the PCB.

In an example, the first section of the PCB has electronic components attached to both the inner surface and the outer surface and the second section of the PCB has electronic components attached only to the inner surface of the PCB.

In an example, the apparatus further includes a carrier that is attached to the PCB and that includes attachment features.

In an example, the first LED bank includes multiple pairs of LEDs, and wherein the second LED bank includes multiple pairs of LEDs, and wherein a first LED of each pair is driven by a first driver signal and a second LED of each pair is driven by a second driver signal.

In an example, the first LED bank includes multiple pairs of LEDs, and wherein the second LED bank includes multiple pairs of LEDs, and wherein at least one pair of LEDs in each LED bank includes LEDs configured to emit different wavelengths of electromagnetic energy.

In an example, the first LED bank includes multiple pairs of LEDs, wherein a first pair of LEDs is configured to emit electromagnetic energy in IR and red bands, and a second pair of LEDs is configured to emit electromagnetic energy in IR and green bands, and the second LED bank includes multiple pairs of LEDs, wherein a first pair of LEDs is configured to emit electromagnetic energy in IR and red bands, and a second pair of LEDs is configured to emit electromagnetic energy in IR and green bands.

A method for producing a finger wearable device is also disclosed. The method involves attaching an inner circular plastic piece to an outer circular metal piece by mating attachment features at each of two opposing ends of the inner circular plastic piece with each of two opposing ends of the outer circular metal piece, wherein the attaching involves at least one of spreading the outer circular metal piece and compressing the inner circular plastic piece to mate the attachment features of the inner circular plastic piece with the attachment features of the outer circular metal piece, wherein upon attachment of the inner circular plastic piece to the outer circular metal piece, a cavity is formed between the inner circular plastic piece and the outer circular metal piece, and wherein sensor electronics are located within the cavity, and after the inner circular plastic piece is attached to the outer circular metal piece, injecting an encapsulant within the cavity.

In an example, the encapsulant is injected into the cavity via a hole in the outer circular metal piece.

In an example, the encapsulant is injected into the cavity via a gap between the inner circular plastic piece and the outer circular metal piece.

In an example, the encapsulant is injected into the cavity via a first hole in the inner circular plastic piece and gas is exhausted from a second hole in the inner circular plastic piece.

In an example, the first hole and the second hole are at opposing ends of the inner circular plastic piece.

In an example, the encapsulant is injected into the cavity via an injection hole in the inner circular plastic piece and gas is exhausted from an exhaust hole in the inner circular plastic piece.

In an example, the injection hole and the exhaust hole are at opposing ends of the inner circular plastic piece.

In an example, the injection hole and the exhaust hole are located in a sidewall of the inner circular plastic piece.

In an example, the encapsulant is injected into the cavity via a first hole in the inner circular plastic piece while a vacuum is created in the cavity from a second hole in the inner circular plastic piece.

In an example, the first hole and the second hole are at opposing ends of the inner circular plastic piece.

In an example, compressing the inner circular plastic piece involves moving the two opposing ends of the inner circular plastic piece closer to each other.

In an example, compressing the inner circular plastic piece involves applying forces to move the two opposing ends of the inner circular plastic piece closer to each other and then releasing the forces after the inner circular plastic piece is moved within the outer circular metal piece.

In an example, compressing the inner circular plastic piece involves moving the two opposing ends of the inner circular plastic piece closer together.

In an example, compressing the inner circular plastic piece involves deforming the circularity of the inner circular piece.

In an example, spreading the outer circular metal piece involves moving the two opposing ends of the outer circular metal piece farther away from each other.

In an example, spreading the outer circular metal piece involves deforming the circularity of the outer circular metal piece.

Another method for producing a finger wearable device is disclosed. The method involves attaching sensor electronics to an outer surface of an inner circular plastic piece, attaching the inner circular plastic piece to an outer circular metal piece by mating attachment features at each of two opposing ends of the inner circular plastic piece with each of two opposing ends of the outer circular metal piece, wherein the attaching involves at least one of spreading the outer circular metal piece and compressing the inner circular plastic piece to mate the attachment features of the inner circular plastic piece with the attachment features of the outer circular metal piece, and after the inner circular plastic piece is attached to the outer circular metal piece, injecting an encapsulant within a cavity that is formed between the inner circular plastic piece and the outer circular metal piece to incapsulate the sensor electronics.

In an example, attaching the sensor electronics to the outer surface of the inner circular plastic piece involves inserting battery charging contacts into through holes in the inner circular plastic piece.

In an example, attaching the sensor electronics to the outer surface of the inner circular plastic piece involves aligning two LED banks and a photodetector that are attached to a PCB with bumps on the inner circular plastic piece.

In an example, attaching the sensor electronics to the outer surface of the inner circular plastic piece involves aligning an LED bank that is attached to a PCB with a bump on the inner circular plastic piece and aligning the LED bank underneath a curved battery that is attached to the PCB, wherein the LED bank and the curved battery are separated by the PCB.

In an example, compressing the inner circular plastic piece involves moving the two opposing ends of the inner circular plastic piece closer to each other.

In an example, compressing the inner circular plastic piece involves applying forces to move the two opposing ends of the inner circular plastic piece closer to each other and then releasing the forces after the inner circular plastic piece is moved within the outer circular metal piece.

In an example, compressing the inner circular plastic piece involves moving the two opposing ends of the inner circular plastic piece closer together.

In an example, compressing the inner circular plastic piece involves deforming the circularity of the inner circular piece.

In an example, spreading the outer circular metal piece involves moving the two opposing ends of the outer circular metal piece farther away from each other.

In an example, spreading the outer circular metal piece involves deforming the circularity of the outer circular metal piece.

In an example, the encapsulant is injected into the cavity via a hole in the outer circular metal piece.

In an example, the encapsulant is injected into the cavity via a gap between the inner circular plastic piece and the outer circular metal piece.

In an example, the encapsulant is injected into the cavity via a first hole in the inner circular plastic piece and gas is exhausted from a second hole in the inner circular plastic piece.

In an example, the first hole and the second hole are at opposing ends of the inner circular plastic piece.

In an example, the encapsulant is injected into the cavity via an injection hole in the inner circular plastic piece and gas is exhausted from an exhaust hole in the inner circular plastic piece.

In an example, the injection hole and the exhaust hole are at opposing ends of the inner circular plastic piece.

In an example, the injection hole and the exhaust hole are located in a sidewall of the inner circular plastic piece.

In an example, the encapsulant is injected into the cavity via a first hole in the inner circular plastic piece while a vacuum is created in the cavity from a second hole in the inner circular plastic piece.

In an example, the first hole and the second hole are at opposing ends of the inner circular plastic piece.

Another method for producing a finger wearable device is disclosed. The method involves at least one of attaching sensor electronics to an outer surface of an inner circular plastic piece and attaching the sensor electronics to an inner surface of an outer circular metal piece, attaching the inner circular plastic piece to the outer circular metal piece by mating attachment features at each of two opposing ends of the inner circular plastic piece with each of two opposing ends of the outer circular metal piece, wherein the attaching involves at least one of spreading the outer circular metal piece and compressing the inner circular plastic piece to mate the attachment features of the inner circular plastic piece with the attachment features of the outer circular metal piece, and after the inner circular plastic piece is attached to the outer circular metal piece, injecting an encapsulant within a cavity that is formed between the inner circular plastic piece and the outer circular metal piece to incapsulate the sensor electronics.

Another example of a finger wearable device for health monitoring, the finger wearable device is disclosed. The device includes an outer circular metal piece, the outer circular piece having two opposing ends, an inner circular plastic piece, the inner circular piece having two opposing ends, a PCB and electronic components connected to the PCB, the PCB and electronic components encapsulated in a cavity between the outer circular metal piece and the inner circular plastic piece, wherein the outer circular metal piece and the inner circular plastic piece include attachment features that are complementary to each other and configured to enable the outer circular metal piece and the inner circular plastic piece to mate together at the attachment features such that the outer circular metal piece and the inner circular plastic piece are held together by the attachment features and such that the outer circular metal piece and the inner circular plastic piece create the cavity within which the PCB and electronic components are encapsulated, and an encapsulant in the cavity between the outer circular piece and the inner circular piece.

In an example, the device further includes a first hole in the inner circular plastic piece and a second hole in the inner circular plastic piece.

In an example, the first hole is located at a first one of the two opposing ends of the inner circular plastic piece and the second hole is at a second one of the opposing ends of the inner circular plastic piece.

In an example, the device further includes an injection hole in the inner circular plastic piece and an exhaust hole in the inner circular plastic piece.

In an example, the injection hole is located at a first one of the two opposing ends of the inner circular plastic piece and the exhaust hole is at a second one of the opposing ends of the inner circular plastic piece.

In an example, the encapsulant is injected into the cavity through the injection hole and gas is exhausted through the exhaust hole.

In an example, the encapsulant is injected into the cavity through the injection hole while a vacuum is created in the cavity through the exhaust hole.

In an example, the injection hole and the exhaust hole are located in a sidewall of the inner circular plastic piece.

The connections as discussed herein may be any type of connection suitable to transfer signals or power from or to the respective nodes, units, or devices, including via intermediate devices. The connections may be illustrated or described in reference to being a single connection, a plurality of connections, unidirectional connections, or bidirectional connections. However, different embodiments may vary the implementation of the connections. For example, separate unidirectional connections may be used rather than bidirectional connections and vice versa. Also, a plurality of connections may be replaced with a single connection that transfers multiple signals serially or in a time multiplexed manner. Likewise, single connections carrying multiple signals may be separated out into various different connections carrying subsets of these signals. The term “coupled” or similar language may include a direct physical connection or a connection through other intermediate components even when those intermediate components change the form of coupling from source to destination.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A finger wearable device for health monitoring, the finger wearable device comprising:
an outer circular piece, the outer circular piece having two opposing ends;
an inner circular piece, the inner circular piece having two opposing ends;
sensor electronics including a printed circuit board (PCB) and electronic components connected to the PCB, the sensor electronics encapsulated in a cavity between the outer circular piece and the inner circular piece;
wherein the outer circular piece and the inner circular piece include attachment features that are complementary to each other and configured to enable the outer circular piece and the inner circular piece to mate together at the attachment features such that the outer circular piece and the inner circular piece are held together by the attachment features and such that the outer circular piece and the inner circular piece create the cavity within which the sensor electronics are encapsulated; and
an encapsulant in the cavity between the outer circular piece and the inner circular piece.

2. The finger wearable device of claim 1, wherein:
the attachment features of the inner circular piece are located at the two opposing ends of the inner circular piece; and
the attachment features of the outer circular piece are located at the two opposing ends of the outer circular piece.

3. The finger wearable device of claim 1, wherein:
the attachment features of the inner circular piece are located at the two opposing ends of the inner circular piece;
the attachment features of the outer circular piece are located at the two opposing ends of the outer circular piece; and
the attachment features at the two opposing ends of the inner circular piece include protrusions that create ridges.

4. The finger wearable device of claim 1, wherein:
the attachment features of the inner circular piece are located at the two opposing ends of the inner circular piece;
the attachment features of the outer circular piece are located at the two opposing ends of the outer circular piece;
the attachment features at the two opposing ends of the inner circular piece include protrusions that create ridges; and
the attachment features at the two opposing ends of the outer circular piece include cavities to receive the ridges of the protrusions of the inner circular piece.

5. The finger wearable device of claim 1, wherein the attachment features include attachment features at the two opposing ends of the outer circular piece and attachment features at the two opposing ends of the inner circular piece.

6. The finger wearable device of claim 1, wherein:
the attachment features of the inner circular piece include protrusions that create ridges at the two opposing ends of the inner circular piece; and
the attachment features of the outer circular piece include surfaces at the two opposing ends of the outer circular piece that mate with the ridges at the opposing ends of the inner circular piece.

7. The finger wearable device of claim 1, wherein the encapsulant is injected into the cavity after the outer circular piece and the inner circular piece are mated together.

8. The finger wearable device of claim 1, wherein the encapsulant is injected into the cavity through a hole in the inner circular piece.

9. The finger wearable device of claim 1, wherein the encapsulant is injected into the cavity through a hole in the outer circular piece.

10. The finger wearable device of claim 1, further comprising an injection hole in the inner circular piece and an exhaust hole in the inner circular piece.

11. The finger wearable device of claim 10, wherein the injection hole is located at a first one of the two opposing ends of the inner circular piece and the exhaust hole is at a second one of the opposing ends of the inner circular plastic piece.

12. The finger wearable device of claim 11, wherein the encapsulant is injected into the cavity through the injection hole and gas is exhausted through the exhaust hole.

13. The finger wearable device of claim 11, wherein the injection hole and the exhaust hole are located in a sidewall of the inner circular piece.

14. The finger wearable device of claim 1, wherein the PCB includes a first section and a second section, wherein the second section is thinner than the first section and wherein the first section has electronic components attached on both an inner side and an outer side of the PCB and the second section has electronic components attached on only an inner side of the PCB.

15. The finger wearable device of claim 1 wherein the electronic components include a curved battery, a photodetector, and two LED banks connected to the PCB, wherein the photodetector is attached to the PCB between the two LED banks and one of the two LED banks is attached at an inner surface of the PCB directly opposite from the curved battery and separated from the curved battery by the PCB.

16. The finger wearable device of claim 1, wherein the inner circular piece has two through holes that pass from the inner surface to the outer surface, and wherein battery charging contacts are located within the two through holes, the battery charging contacts being electrically connected to a curved battery in the cavity.

17. The finger wearable device of claim 1, wherein:
the inner circular piece has ridges that form a channel at an outer surface of the inner circular piece; and
the outer circular piece has ridges that from a channel at an inner surface of the outer circular piece;
wherein the ridges of the inner circular piece are mated with the ridges of the outer circular piece to create the cavity,
wherein the ridges of the inner circular piece are mated with the ridges of the outer circular piece around a circumference of the inner circular piece and a circumference of the outer circular piece.

18. The finger wearable device of claim 1, wherein the inner circular piece includes a component bay to receive an electronic component of the sensor electronics, wherein the component bay includes a recess below a plane of an outer surface of the inner circular piece.

19. The finger wearable device of claim 1, wherein the inner circular piece includes an alignment feature on an outer surface of the inner circular piece, wherein the alignment feature is a protrusion on the outer surface of the inner circular piece.

20. A finger wearable device for health monitoring, the finger wearable device comprising:
an outer circular metal piece, the outer circular piece having two opposing ends;
an inner circular plastic piece, the inner circular piece having two opposing ends;
a printed circuit board (PCB) and electronic components connected to the PCB, the PCB and electronic components encapsulated in a cavity between the outer circular metal piece and the inner circular plastic piece;
wherein the outer circular metal piece and the inner circular plastic piece include attachment features that are complementary to each other and configured to enable the outer circular metal piece and the inner circular plastic piece to mate together at the attachment features such that the outer circular metal piece and the inner circular plastic piece are held together by the attachment features and such that the outer circular metal piece and the inner circular plastic piece create the cavity within which the PCB and electronic components are encapsulated; and
an encapsulant in the cavity between the outer circular piece and the inner circular piece.
